(19) United States
(12) Patent Application Publication
(10) Pub. No.: US 2004/0162511 A1
(43) Pub. Date: Aug. 19, 2004

(54) Surgical cast venting device with padding

(76) Inventor: Alessandro Barberio, Toronto (CA)

Correspondence Address: Thomas N. Young, YOUNG & BASILE P.C., Suite 624, 3001 West Big Beaver Road, Troy, MI, 48084, US

(21) Appl. No.: 10/366,188

(22) Filed: Feb. 13, 2003

(51) Int. Cl. A61F 5/00

(52) U.S. Cl. 602/14

(57) ABSTRACT

A venting device adapted for placement around body part prior to application of a surgical cast including a piece of fabric and aerating devices affixed to the inner surface of the fabric. Padding material preferably made of ethyl vinyl acetate (EVA) or PLASTAZOTE and is affixed upon at least some of the aerating devices and is positioned to be disposed between the body part and each aerating device. Padding material may form a plurality of padding envelopes into which aerating devices may be inserted. Aerating devices are preferably elongate perforated tubular members. Padding material may be elicoidally wrapped along the length of the tubular members. Padding material can have perforations some of which align with perforations in the tubular members. To assist the tubular members to bear the weight of the body part, rigid cylinders can be placed over portions of the tubular members and stoppers at the end of tubes can be provided. A preferred device is stretchable over the body part and a layer of the tubes is arranged elicoidally upon the inner surface of the fabric. The padding allows enhanced comfort for surgical cast venting devices which are applied directly to the skin.

### BACKGROUND OF THE INVENTION

This invention relates to devices for venting a surgical cast.

In the attempt to solve the problem of supplying ventilation to skin under a surgical cast, a variety of different structures and methods have been disclosed. For example, according to my laid open Canadian patent application No. 2,254,492 filed Nov. 25, 1998, a surgical cast venting device can be constructed in the form of a strip. The strip is elongate and made of porous fabric, gauze or casting material. Aeration devices such as plastic or latex tubing are attached at regular intervals along the woven fabric or gauze material.

Disclosed in the same Canadian application is a surgical cast venting device comprising an elongate porous woven fabric strip, a number of flexible, elongate tubes with holes distributed along their respective lengths, these tubes being distributed along the length of and attached to the fabric strip on one side thereof, each tube extending lengthwise in a direction substantially parallel to this one side. The elongate strip is suitable for winding around part of a human body or an animal's body prior to application of a surgical cast over this part of the body.

Another embodiment of a surgical cast venting device disclosed in the aforementioned Canadian application comprises an elongate, flexible strip of thin porous material and a number of relatively short, elongate tubing members distributed over and mounted on the elongate strip on one side thereof. The length of each tubing member extends substantially parallel to this one side. The elongate strip with the tubing members is adapted for winding around part of a human body or an animal's body prior to application of a surgical cast over this part of the body.

In a further embodiment of this venting device, there are perforation holes along the length of the tubes. Also, the tubes are round or oval shaped in cross-section.

In order to use the device, the elongate strip is wound around a human's or animal's arm or leg, such that the side of the strip to which the tubes are applied completely covers an area of dressed skin. Once the strip has been wound, it can be secured in place by Velcro strips, for example. The plaster is applied over the venting device. The cast then hardens and the cast is then completed. The natural movement of a person's or animal's muscles under the cast will serve to vent the cast if this venting device is properly arranged under the cast.

In my U.S. patent application Ser. No. 09/533,214 filed Mar. 23, 2000, there is disclosed a surgical cast venting device comprises an elongate porous fabric strip having two opposite ends, having two opposite, longitudinal side edge sections, and having inner and outer surfaces. Aerating devices are affixed to and located on the inner surface. A multiple hook array fastener arrangement extends along and is secured to a first of the longitudinal side edge sections and is adapted to detachably secure this first longitudinal side edge section to a second of the longitudinal side edge sections when said fabric strip is wound around part of the human or animal body prior to application of a surgical cast over this part of the body.

Preferably the elongate strip is tapered at the ends. With respect to the tubes on the strip, they can be attached at a slight angle to a line extending transversely across the strip. One third of the width of the strip is covered by the tubes, except near the ends of the strip.

Shown in the aforementioned applications is the addition of sponge strips partially surrounding portions of the tubing. Disclosed are two sponge strips located at opposite ends of each tube and a central strip positioned midway between the end strips. These strips can provide more comfort to the user and create a gap between the plastic tubing and users skin or cloth cover or sleeve if previously applied to the skin. These strips may not prevent the portions of the tube between the strips from contacting the skin and thus may not fully enhance the comfort of the user. Furthermore, since these strips are not perforated, they must be sufficiently thin so as to not interfere with perforation holes which may be present in the tubing and which are designed to aid in the aeration of the tubing. These strips are not capable of being placed over the entirety of the tubing without potentially significantly interfering with aeration of the tubing.

In my further previous U.S. patent application Ser. No. 09/476,106, filed Jan. 3, 2000, and incorporated herein by reference, there is disclosed a surgical cast venting device including a stretchable piece of fabric material having at least some elasticity, having two opposite ends and two opposite side edges, and having inner and outer surfaces. At least one aerating device is affixed to and located on the inner surface. Hook and loop type flexible fastener strips are affixed to the two opposite side edges and are arranged for detachable connection to one another in order to form the piece of fabric material into a tubular configuration or a portion of a tubular configuration. This venting device is adapted for placement around part of a human body or animal body prior to application of a surgical cast over this part of the body.

To increase the comfort of the user, the venting devices disclosed in the above mentioned previous applications are generally applied after the application of a thin breathable cloth or jersey material in a known manner to at least portions of the injured body part and prior to the application of the cast. The present invention proposes the inclusion of padding material between the aeration devices and the body part in order to provide improved comfort to the user. This padding may be affixed to significant portions of the aerating devices to enhance comfort of the user.

### SUMMARY OF THE INVENTION

It is an object of the invention to provide an improved surgical cast venting device.

According to one aspect of the invention there is provided a surgical cast venting device comprising a fabric strip, having two opposite ends defining a length therebetween and having inner and outer surfaces, the strip being applicable to a part of a human or animal body prior to application of a surgical cast over the body part, the inner surface facing said body part when said strip is applied thereto. A plurality of aerating devices are affixed to the inner surface and padding material is affixed to the aerating devices and positioned such that the padding material is disposed between the aerating devices and the body part when said strip is applied to the body part.

Preferably the aerating devices comprise a substantial number of flexible tubular members each having two ends and being adhesively bonded to the fabric strip. Preferably the tubular members have perforation holes formed through the side thereof. Preferably the padding material is ethyl vinyl acetate (EVA) and is perforated so as to allow air to pass therethrough. Preferably at least some of the perforations in the tubular members align with perforations in the padding such that air flow in the device is maximized. Preferably tubes are positioned adjacent to each other. Padding may be attached to the tubes in any suitable manner. For example, padding may be wrapped around each tube or padding may be wrapped around groups of tubes in a padding envelop.

The padding material may be elicoidally wrapped along the length of the each tube and each tube may include means to assist the tube to support the weight of the body part, such as a tube stopper inserted at an end of the tube or rigid cylinders to be inserted over portions of the tube. Aerating devices may be included over a portion of or the entirety of the inner surface.

According to a further aspect of the invention, a plurality of padding envelopes each comprising a hollow central portion are positioned upon the inner surface of the strip and aerating devices are positioned within the padding envelopes, the padding envelopes being positioned adjacent to each other, preferably without any gaps therebetween. In one version of the invention, a thin layer of heat managing and perspiration absorbing material such as cotton, stockingnette or Comfortem (TM) foam is positioned between the limb and the padding material.

In accordance with a further aspect of the invention there is provided a surgical cast venting device comprising a stretchable piece of porous fabric material having elasticity and substantially tubular configuration, the piece having an inner surface shaped and sized to stretch and snugly fit over a body part prior to the application of a surgical cast, and this piece having two opposite ends. At least one layer of a plurality of flexible tubular members, distributed over, and affixed to the inner surface in an elicoidal pattern is provided. The tubular members are open ended and padding material affixed to at least some of said tubular members such that the padding material is disposed between the tubular members and the body part when the venting device is fit over the body part. Preferably tubes are aligned adjacent to each other. Padding may be wrapped around each tube or padding may be wrapped around groups of tubes.

In accordance with a further aspect of the invention, there is provided a surgical cast venting device comprising a porous piece of fabric material having at least some elasticity and a substantially tubular configuration, the piece having two opposite ends, and an inner surface. At least several, separate elongate plastic tubular members are distributed over and affixed to a portion of the inner surface. The tubular members each are open ended at at least one end and have at least several ventilation holes formed in the tubular side thereof. The tubular members extend substantially from one of the opposite ends to the other end and padding material is affixed to portions of at least some of the tubular members. The padding material includes perforation holes therethrough, some of which being aligned with the ventilation holes. The venting device is adapted for placement around part of a human body or animal body prior to application of a surgical cast over this part of the body.

In accordance with yet another aspect of the invention there is provided a surgical cast venting device comprising an elongate flexible strip of porous fabric material for winding around part of a human or animal body prior to application of a surgical cast, a number of elongate flexible tubing members distributed over and attached to the elongate strip on a portion of one side thereof and padding material attached to the tubing members and positioned such that the padding is disposed between the tubing members and the body part when the elongate strip is wound around this body part.

The tubing members may be relatively short and the length of each tubing member extends substantially parallel or perpendicular to the length of the one side. The elongate strip may be tapered at opposite ends thereof and the tubes may be attached at a small acute angle to a transverse line perpendicular to a longitudinal centreline of the elongate strip. Preferably tubes are positioned adjacent to each other.

The tubes may cover only a portion of the strip, leaving a portion uncovered by the tubes. Padding may be adhered to the tubes in any suitable manner. Means for securing the fabric strip in place after the strip has been wound around the body is also provided.

An advantage provided by this invention is that the inclusion of padding will enhance the comfort of the various embodiments and devices described before applying the cast or other means of immobilization and without substantially hindering airflow.

### BRIEF DESCRIPTION OF THE DRAWINGS

FIG. 1 is a plan view showing my surgical cast venting strip as described in Canadian patent application no. 2,254,492 with padding added;

FIG. 1A is a plan view of the venting strip similar to that of FIG. 1 showing aerating devices in schematic form and the portion of the strip without aerating devices removed;

FIG. 1B is a plan view of a venting strip similar to that illustrated in FIG. 1A utilizing tubes as aerating devices;

FIG. 2 is a perspective view of this surgical cast venting strip being unrolled with padding added;

FIG. 3 is a side view of the cast venting device with padding added applied and wrapped around a human leg;

FIG. 4 is a cut away elevational view of the surgical cast venting device covered by a hardened cast, the latter being partially cut away for sake of illustration;

FIG. 5 is a side view of the surgical cast venting device of FIG. 1 and showing an air hose being applied to vent the device;

FIG. 6 is a side view of an upper portion of the earlier surgical cast venting device showing a hair dryer being used to vent the device;

FIG. 7 is a side view of an alternative construction for the plastic tubing members as described in my laid open Canadian application;

FIG. 8 is an end view of the alternative tubing member of FIG. 7;

FIG. 9 is a plan view similar to FIG. 1 but showing an alternate embodiment of the cast venting strip described in my earlier laid open Canadian application with padding added;

FIG. 10A is a plan view of the outer surface of another version of a surgical cast venting strip;

FIG. 10B is a plan view of the inner surface of a venting strip of FIG. 10A showing the addition of padding;

FIG. 11 is a plan view of an outer surface of an alternate version of the cast venting strip;

FIG. 12 is a plan view of the outer surface of three separate components that can be connected together to form a long surgical cast venting strip with central portions of the two end components being omitted to indicate indefinite length;

FIG. 13a is a detail sectional view illustrating one preferred method for attaching adjacent longitudinal edges of the venting strip using hook and loop type fastener arrangements;

FIG. 13b is a detail sectional view similar to FIG. 13b but illustrating an alternate form of longitudinal edge construction using hook and loop type fastener arrays;

FIG. 14 is a cross-sectional detail of a longitudinal edge construction for the venting strip illustrating one form of plastic edge support for a hook or loop type fastener array (not shown);

FIG. 15 is a cross-sectional detail similar to FIG. 14 but showing another form of plastic edge support;

FIG. 16 is a further cross-sectional detail similar to FIG. 14 but showing a further version of the plastic edge support;

FIG. 17 is a detail edge view showing a portion of another embodiment of a venting device, this embodiment having aerating devices in the form of a layer of non-woven, plastic threads and including padding affixed thereto;

FIG. 18 is a top view of the small section of the venting device shown in FIG. 17 showing padding affixed thereto;

FIG. 19 is a plan view similar to FIG. 18 but showing a further manner of constructing a venting device with padding affixed thereto;

FIG. 20 is a detail view of a section of still another embodiment of venting device, this version employing plastic ring members with padding affixed thereto;

FIG. 21 is a plan view of the section of the venting device shown in FIG. 20 with padding affixed thereto;

FIG. 22 is an edge view of a small section of a further embodiment of venting device, this version employing corrugated plastic members with padding affixed thereto;

FIG. 23 is a plan view of the small section of the venting device shown in FIG. 22 with padding affixed thereto;

FIG. 24 is an edge view of part of another embodiment of the venting device, this version employing plastic members with corrugated tops with padding affixed thereto;

FIG. 25 is a plan view of the section of the venting device shown in FIG. 24 with padding affixed thereto;

FIG. 26 is an edge view of still another embodiment of the venting device, this version employing a grid of plastic members with a number of bumps formed on their top surfaces with padding affixed thereto;

FIG. 27 is a plan view of the section of the venting device shown in FIG. 26 with padding affixed thereto;

FIG. 28 is an edge view of part of another venting device, this embodiment employing a grid work of small plastic members having a bump formed in each of their top surfaces with padding affixed thereto;

FIG. 29 is a detail view of the section shown in FIG. 28;

FIG. 30 is a detail plan view of part of an inner surface of a further form of venting device with padding affixed thereto;

FIG. 31 is an edge view illustrating plastic tubular members having an oval cross-section, such members having padding affixed thereto and being usable in still another version of the invention;

FIG. 32 is a detail view showing a portion of the inner surface of a venting device similar to that shown in FIG. 2, this portion being shown flat for ease of illustration and with padding affixed thereto;

FIG. 33 is a detail edge view of a portion of the venting device of FIG. 1 or FIG. 2 illustrating the manner in which the tubular members can be adhered to the porous fabric;

FIG. 34 is a side detail view illustrating another form of tube that can be used for the venting device with padding affixed thereto;

FIG. 35 is an end view illustrating a section of a venting device made with the tubes of FIG. 34 with padding affixed thereto;

FIGS. 36A, 36B, 36C and 36D show alternative ways of padding of the tubes shown in the Figures above;

FIGS. 37A to 37D also show alternative ways of padding the tubes used in the venting devices of the invention;

FIGS. 37E to 37L show various embodiments of padding envelopes use to house aerating devices of the invention;

FIGS. 38 and 38A shown the padding of a kinkless tube with elicoidally (spirally) wrapped padding;

FIGS. 39A, 39B, 40A, 40B are illustrations of tube stoppers which are insertable into the ends of tubes and help to prevent padding material from sliding once placed on the tubes and help to support the weight of the body part;

FIG. 41A is a perspective view of a tube stopper being placed in an end of an aeration tube;

FIG. 41B shows how the weight of the body appendage will cause the tube stopper to deform;

FIG. 42A shows a metal or plastic cylinder member for additional support to a tube;

FIG. 42B shows the use of several of these cylinder members on a plastic tube;

FIG. 43A is a perspective view of an ellicoidal arrangement of tubes with padding attached;

FIG. 43B is a schematic representation of an aspect of the arrangement shown in FIG. 43A and illustrating certain significant measurements;

FIGS. 44, 45 and 46 are side views showing further embodiments of venting devices with addition of padding thereto; and

FIG. 47 is a schematic drawing showing the inclusion of aerating devices with padding on a portion of the strip of fabric to be applied to the body part.

### DETAILED DESCRIPTION OF PREFERRED EMBODIMENT

As is shown in the figures and as will be described below, padding 330 is added to various embodiments of cast venting devices. This padding and optional additional layers of heat and perspiration absorbing material will enhance comfort of the venting devices when applied directly on the skin. The padding 330 can be attached to any of the below described aerating devices by means of a suitable adhesive that will firmly adhere to both the padding material and the adjacent device or strip such as medical adhesive material. Furthermore, padding may be adhered by means of inoxydable staples, surgical tape, threading or velcro.

The padding must be of sufficient density such that it reduces the pressure exerted on the leg, arm, or torso when the cast surrounds such body parts because the padding absorbs a good portion of the pressure.

The padding material used may be NORA (Trademark)-EVA (ethylvinyl acetate) LUNAIMED perforated material having a hardness of Shore A=18 degrees. This material is sometimes referred to herein as simply “EVA”. It is estimated that 1.0 square centimeter of the improved venting device that uses this padding should support 50 grams of weight without bending. The EVA material will add more supportive resistance to the tube. The padding material can be comprised of different materials such as EVA, PLASTAZOTE, CONFORTEM (trade-mark), the latter being a foam for thermal management that can be used for medical devices and surfaces.

Alternately, padding material can comprise a combination of EVA, cotton and foam, which materials can be used to provide either a cooling or a warming effect for the patient. Also, the padding may be an EVA-PLASTAZOTE combination, or perforated PLASTAZOTE.

As is shown in the Figures described below, padding, preferably NORA (Trademark)-EVA, is applied such that it will be disposed between the various aeration devices disclosed herein and the injured human or animal body part. The padded aeration devices described below can also be used in special support boots, under various known braces for body parts, as well as under casts. These venting devices are suitable for use on both humans and animals on which casts or braces are currently used.

Although, padding material is preferably NORA(Trademark)-EVA (available from National Shoe), any suitable padding material may be used. Preferably the padding material will have the following characteristics: (1) should be soft on the skin to aid in comfort, (2) should be elastic to allow for molding, (3) resilient such that it will absorb pressure when the cast is tightened firmly around the injured body parts, (4) perforated and/or porous such that it will allow generous aeration (5) hypoallergenic and non-toxic and (6) practical. The preferred padding will allow the user to subject the cast to water without causing undue retention of water beneath the cast, as is likely to occur where a cotton layer is present beneath the cast.

In order to reduce the likelihood of the padding material sliding relative to the skin due to perspiration or other moisture developing between the skin and padding, there may be provided adjacent to the skin, a layer of padding material which is softer than the preferred padding material and which may include a course surface for contacting and adhering to the skin. Alternately, the padding material itself may be soft and include such a course surface for adherence to the skin.

For padding applied to the various aeration devices such as those shown in the Figures below, the padding preferably includes perforations 350 (see FIG. 18 for example) which may align with perforations (or holes) in the aerating devices, or which generally aid in the ventilation of the device and therefore do not hinder and may in fact enhancing aeration functionality. Although the Figures below show padding 330 only covering portions of each tube or other aeration device, padding may be affixed to portions of the, tube (or other aeration devices) or it may cover the entirety of all tubes (or other aeration devices) on the venting device or it may cover selected tubes or aeration devices on the venting device. There may be provided one layer of padding 330 or there may be provided multiple layers of padding. Although the figures herein generally show one layer of padding 330 disposed upon the aerating devices, in order to improve the comfort of the user, and absorb additional pressure of the casting on the skin of the user, an extra layer or extra layers of padding may be included between the venting strips and aerating devices. Alternately, such extra layer may be attached upon the padding and situated between the aerating device and the skin. Further alternately, to enhance comfort, a thin layer of material which absorbs perspiration and manages heat, such as cotton, stockingnette or Comfortem (TM) foam (or other comparable heat managing materials) maybe adhered and positioned on the padding. With the presence of such perspiration and heat absorbing material directly on the padding of the device, the need for the additional step of placing such heat managing and perspiration absorbing materials on the limb prior to placement of the casting device is eliminating thereby saving time and health care manpower.

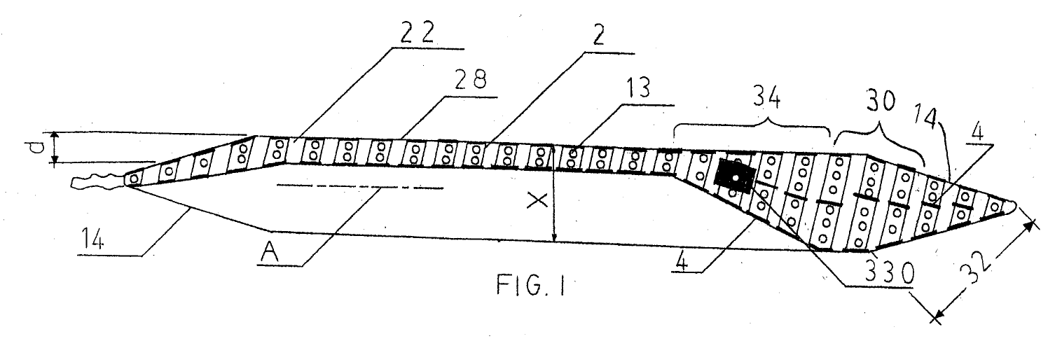

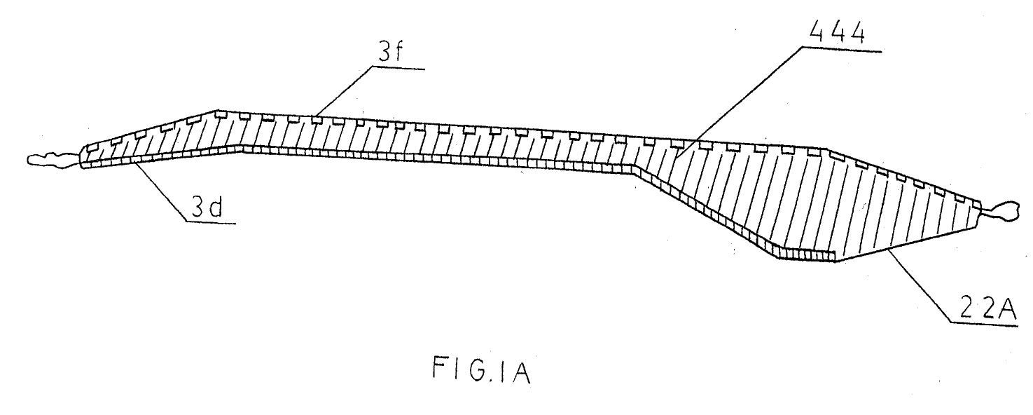

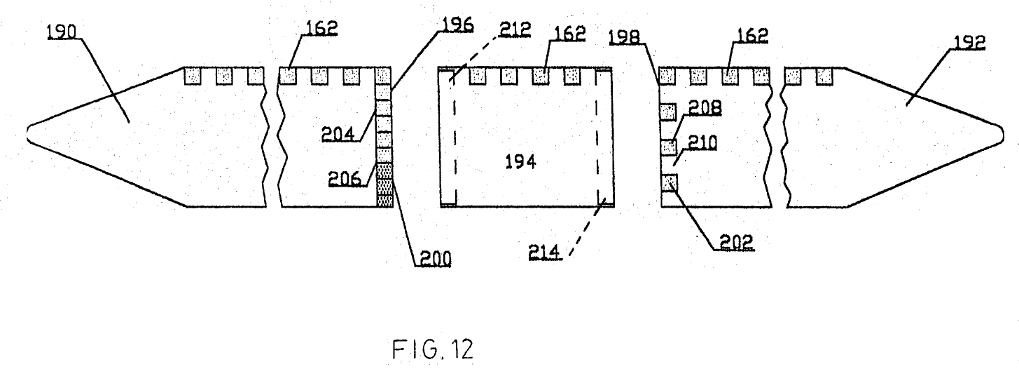

Shown in FIG. 1 is a form of surgical cast venting device 1 that includes an elongate strip 22 of porous fabric material, preferably a woven fabric. The device 1 is shown and described in my Canadian patent application Serial no. 2,254,492 which was laid open on or about May 25, 2000. One suitable form of material is a gauze material similar to that used to make bandages. The length of strip 22 can vary and will depend to some extent on the particular cast that it is to be used with. In a situation where strip 22 proves to be of insufficient length, one or more of the strips can be attached end to end, for example, by means of Velcro fastening strips as illustrated in FIG. 12. A number of relatively short, elongate plastic tubing members 2 are distributed over and attached to the elongate strip on one side 24 thereof. The tubes are preferably 7 to 10 mm in external diameter and can be open ended. Preferably the plastic from which the tubes are made is reasonably flexible so that the cast venting device will be comfortable when applied in the manner described hereinafter. The elongate strip with the attached tubing members is adapted for winding around part of a human body (such as an arm or leg) or an animal's body that has been injured prior to application of a surgical cast over this part of the body.

Preferably plastic tubing members 2 each have a number of small holes 13 distributed along their respective lengths so as to permit the passage of air in and out of the tubing members. These holes 13 can, for example, be arranged in several rows along the length of the tubing members, if desired, in order to provide good ventilation.

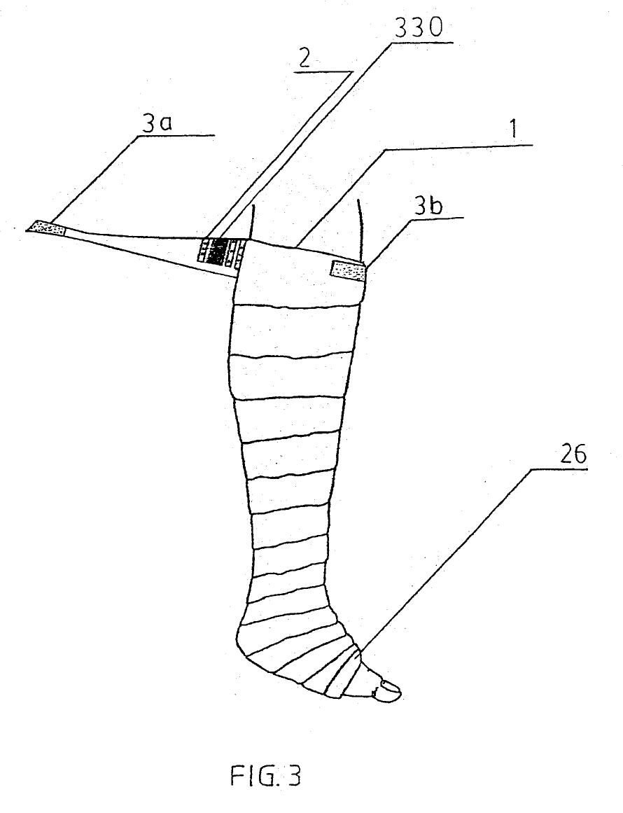

FIG. 3 illustrates how the venting device 1 of the invention can be wrapped around the leg of a human, for example, one that has been broken. The venting device as shown is wrapped first around the foot at 26 and is then wrapped in a partially overlapping manner as shown until the venting device is completely wrapped around the body part. Also note that the side with the plastic tubing members attached thereto is the side facing towards the leg. Padding 330, preferably EVA with perforations 350, may be attached to plastic tubing members such that it will be disposed between the tube and part being surrounded by the venting device. Preferably, at least some of perforations 350 align with holes 13 to aid in aeration of the device.

The wraps formed by the venting device should be arranged so that the tubing members 2 are not wrapped on top of one another. In other words, the tubing members when the cast venting device has been put in place, should be distributed reasonably evenly and in a single layer only over the area to be covered with the cast. In one version of the strip, the distance between adjacent tubes is about equal to the diameter of the tubes themselves but this distance will increase slightly as the strip is stretched.

Although the various embodiments of the invention where the aerating devices comprise tubes are illustrated in the figures herein showing the tubes as being spaced from one another, in certain preferred versions of the invention, tubes or other aerating devices are positioned adjacent to each other such that there is no distance between tubes. Positioning of tubes adjacent to each other enhances comfort of the user. When tubes are spaced from one another, it may be preferable to include an extra layer of padding either between venting device and skin or between the venting device and venting strip to enhance the comfort of the user. Where the venting strip is wrapped loosely around the skin, tubes or other aerating devices may be spaced apart from one another without the inclusion of an extra layer of padding.

To permit application of the venting device in a continuous wrapped manner, this venting device is constructed in the manner illustrated in FIG. 1. Firstly, it will be noted that the elongate strip 22 is tapered at 14 at opposite ends thereof. As shown, each end tapers inwardly in a direction away from the longitudinal center of the strip. Also, for a substantial portion of the length of the elongate strip, the plastic tubing members 2 extend from one long edge 28 of the strip a distance which does not exceed one half the width of the strip, this distance being indicated by the dimension d in FIG. 1. In fact, in the illustrated embodiment, the distance d is about one third the maximum width of the fabric strip from the longitudinal edge 28. This maximum width is denoted x, and can vary depending of the region of the body to which the device is applied. When the strip is to be wound around a straight portion of a leg, x is preferably about 4 inches. When the strip is to be wrapped around a bend such as a knee, x can be somewhat greater, about 4.5 inches.

At one end of the strip the plastic tubes can extend further as shown in FIG. 1, this being the end where the wrapping operation commences. Thus, the tubing at 30 extends substantially the entire width of the strip 22. Also, the tubing at 32 located at the tapered end 14 can extend the entire width of this tapered portion. In the region 34 of the strip that extends between the region 30 and the much longer region where the tubes are of substantially the same short length, the tubes can gradually shorten in length to provide a transition region. At the opposite end, the tubing members can be quite short and extend only part way across the strip. With the tubing members arranged in this manner, each wrap of the venting device (see FIG. 3) can be made to overlap the previous wrap a substantial distance without resulting in the tubing members being laid on top of each other. In other words, if the tubing members extend only one third of the width of the strip for most of the length of the strip, then the uncovered portion 40 of the strip, will overlap the adjacent and underlying wrap of the strip. In this way, the wrapped venting device 1 will form a securely wrapped sleeve about the leg or arm and each wrap of the venting device will be held firmly in place prior to the application of the casting material.

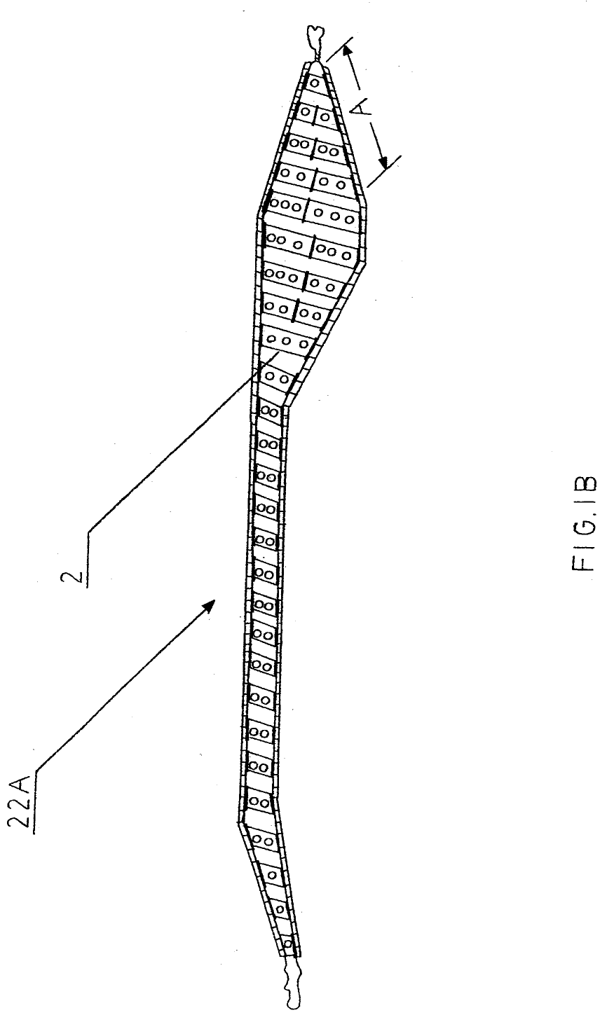

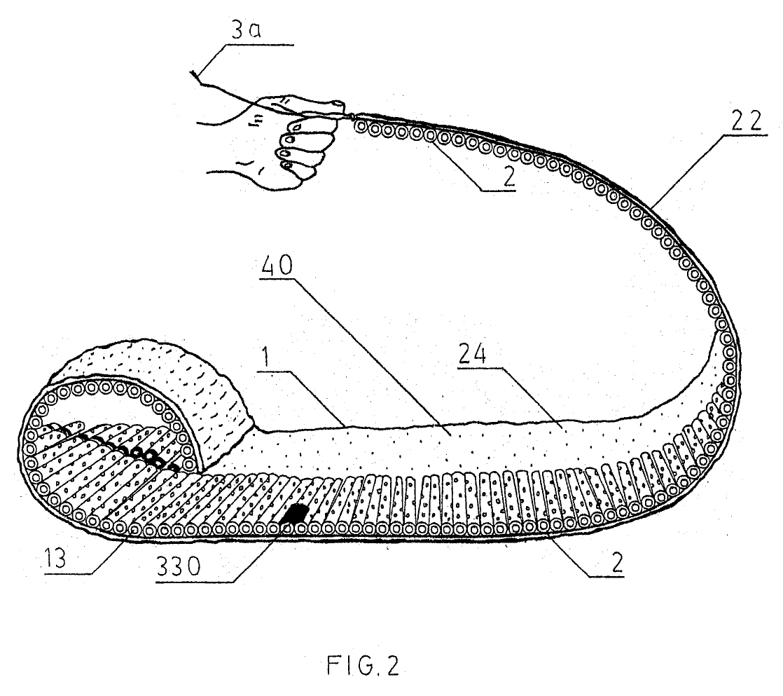

As shown in FIG. 1, the plastic tubing members 2 can be attached or bonded to the strip 22 at a small acute angle to a transverse line (for example, the illustrated line X) perpendicular to a longitudinal central axis of the elongate strip. This central axis is indicated by the dashed line A in FIG. 1. In the alternative, the tubes can be arranged so that their length extend perpendicular to the longitudinal central axis A, this arrangement being shown in FIG. 2.

It is also possible to construct this earlier cast venting device as shown in FIG. 9 using longer plastic tubing members 60 that extend substantially in the longitudinal direction of the fabric strip. These tubing members can be aligned end-to-end in rows as shown and would have sufficient flexibility to readily bend along body contours. Again, the tubing in the region 54 is distributed evenly across the entire width of the fabric strip. Also, the tubing at 52 located in the region of the tapered end is evenly spaced across the entire width of this tapered portion with the tubes parallel to the longitudinal axis. In the region 50 of this device, the tube lengths are gradually shortened to provide a transition region. These longer plastic tubes can also be open ended at their ends 58.

It will be particularly noted from FIG. 9 that the long tube members are interrupted by short spaces between adjacent ends 58. These space interruptions are aligned at an acute angle to the longitudinal centerline of the strip 22. This acute angle is selected and used to give more freedom when the venting device is wrapped around a body part or limb and makes the device easier to wrap.

This cast venting device can be equipped with means for securing the fabric strip in place after the fabric strip has been wound around the body part. Although a variety of securing devices are possible, one form of securing device includes hook and loop type fastening strips attached at one or both ends of the fabric strip. These well known types of fasteners are sold under the VELCRO trade-mark and they are indicated at 3a and 3b in FIGS. 2 and 3. The strip 3a is one half of the fastener system and consists of a strip with a gridwork of small hooks formed thereon as is well known in the fastening art. The strip 3b, which is attached to the strip 22 so that it can be readily engaged by the strip 3a when the wrapping procedure has been completed and can be located a distance from the end of the strip, can be a strip covered with small loops which are easily engaged by the hooks on the strip 3a. Provided the strip 22 is made from a suitable porous fabric material that can be readily engaged by the hooks on the strip 3a, one could, in the alternative, omit the strip 3b and simply attach the strip 3a to the outer side of the strip 22.

Shown in FIG. 1A is a fabric strip 22a which is a variation of the strip of FIGS. 1, 2 and 3, showing aerating devices 444 attached thereto in schematic form. The strip 22a is similar to that illustrated in FIGS. 1, 2 and 3, except that the portion of the strip in FIGS. 1, 2 and 3 which does not include aerating devices has been removed. The shape of strip 22a allows for further efficient wrapping around the desired limb. Although strip 22a may include aerating devices such as tubes 2, it should be understood that the strip 22a may include any of the aerating devices described herein and illustrated in the figures below. Velcro 3d, 3f may be attached along the entirety or portions of the edges of the strip for fastening the strip in place once wound around the body part. In fact, the strip may be fastened in place once wrapped around the body part by any manner, including those fastening devices described in the herein application. Padding material 330 may be adhered to some or all of the aerating devices 444, such that the padding material is disposed between the skin and aerating device. FIG. 1B shows the venting strip illustrated in schematic form in FIG. 1A, utilizing tubes as aerating devices. Note that velcro may be adhered to portions or the entirety of the edges of the strip such that the strip may be fixed in place once wound around the desired body part. For example, portion A may not include velcro.

Other types of securing means (not shown in the drawings) could include metal or plastic snap fasteners or simply cloth or rope tying members. It will be appreciated that the length of the elongate strip 22 can be made adjustable, if desired. One way of making it adjustable is described in detail below.

The plastic tubing members 2 can be attached in a permanent manner to the strip 22 by means of a suitable adhesive. The adhesive should be selected so as to be compatible with both the material of the strip 22 and the plastic from which the tubing is made and should provide a secure bond to both types of material.

As illustrated clearly in FIG. 2, in this particular venting device the elongate tubes 2 are arranged so that their longitudinal central axes are substantially parallel when the fabric strip is laid out flat and straight and the tubes are arranged only a short distance apart from the adjacent tubes, for example, a distance of about the diameter of each tube.

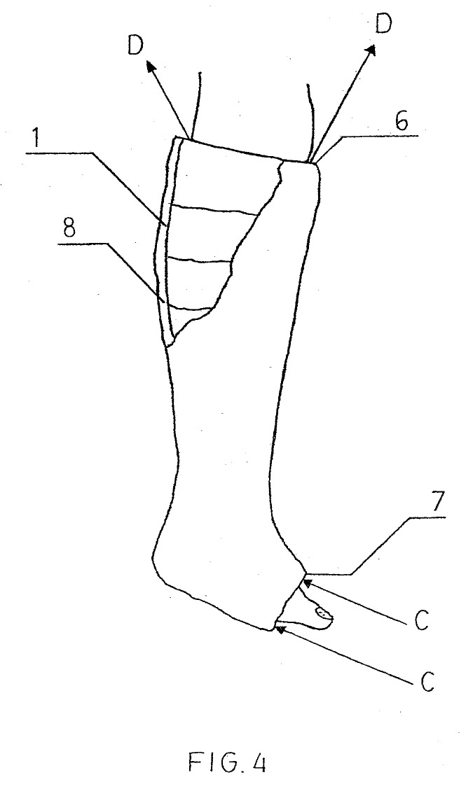

In order to use a venting device 1 without padding 330 such as EVA, the injured body part (after the bone has been reset) is preferably suitably covered with a thin cloth or jersey material in a known manner in order to ensure the comfort of the user. With the addition of padding material 330 in the manner described herein, the venting device 1 can be wrapped around the injured body part and put directly in place since the padding has characteristics which make it comfortable for the user to apply it directly to the skin. It should be understood that comfort may be further enhanced by the addition of a heat managing and perspiration absorbing material layer such as cotton, stockingnette or Comfortem (TM) foam (or other comparable heat managing material) over the skin. As discussed above, the heat and perspiration absorbing material may be positioned directly on the padding positioned between the padding and the limb, thereby eliminating the need to add the additional layer prior to the venting device. Next, the usual casting material 8 is placed over the venting device as illustrated in FIG. 4, Once this casting material has set, a rigid cast is formed about the venting device, thus protecting the injured limb and allowing it to heal. The layer 8 can, for example, be made of a known plaster composition. Note that the cast layer 8 has been omitted from FIGS. 5 and 6 in order to show the underlying venting device 1.

With the use of this particular venting device, it is expected that natural venting or aeration of the skin under the cast will occur simply as a result of normal muscle movement. Because this venting device is made from flexible, pliable material, some limited degree of movement of the muscles in the leg or arm is permitted by this device and this will result in some movement of air into and out of the venting device as indicated by the arrows at C and D in FIG. 4. For example, the muscle movement may cause air to enter at the bottom 7 of the cast as indicated by the arrows C and cause a corresponding amount of air to exit from the top 6 of the cast as indicated by the arrows D.

Further increased venting of the cast can be caused by using the methods illustrated in FIGS. 5 and 6 of the drawings. In FIG. 5, air is being forced into the top end 6 of the venting device by means of an air hose 18 having a nozzle 11 with a narrow end 17. Pressurized air could for example be provided to the hose 18 by means of a suitable compressor attached thereto. Air directed into the top of the venting device at 6 will eventually pass through the entire length of the venting device and exit at the bottom end 7.

FIG. 6 illustrates how a standard hair dryer 9 can be used to force air through my venting device. The hair dryer can be provided with a special outlet attachment 10 with a narrow, flat outer end at 19. As illustrated, air can be again forced into the top end of the venting device by placing the end 19 adjacent this upper end of the venting device and operating the hair dryer. The fast air flow will eventually pass through the length of the venting device and exit through the bottom end thereof.

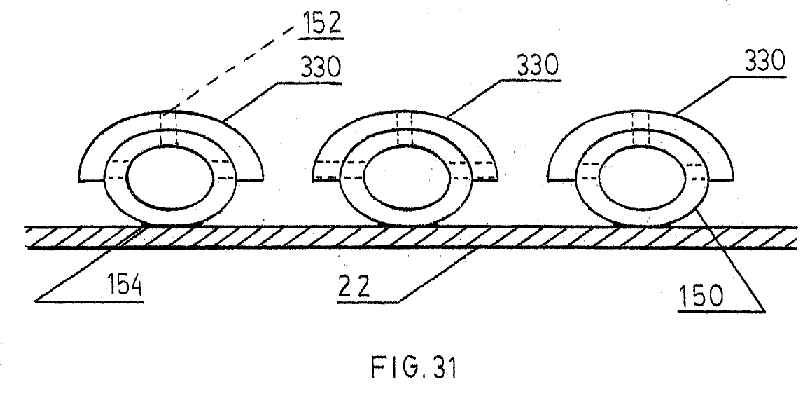

The tubing members 2 may not necessarily be made of a flexible plastic material but they could also be made of other materials such as a flexible latex material. The tubes can have a cross-section other than round and they could, for example, have an oval cross-section as shown in FIG. 31.

In figures discussed below there are illustrated further embodiments of the venting device of the invention which include padding material 330, preferably EVA. The padding 330 shown affixed to the embodiments below, preferably includes perforations 350 which align with at least some of the holes or perforations in some of the embodiments of the aeration devices.

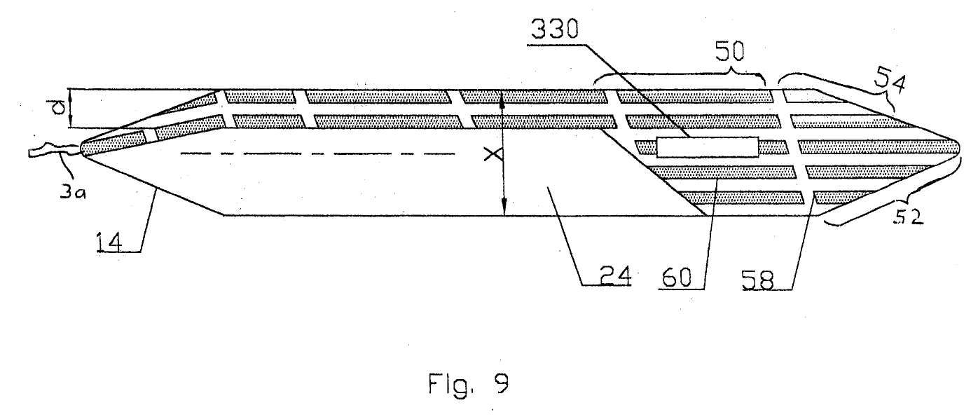

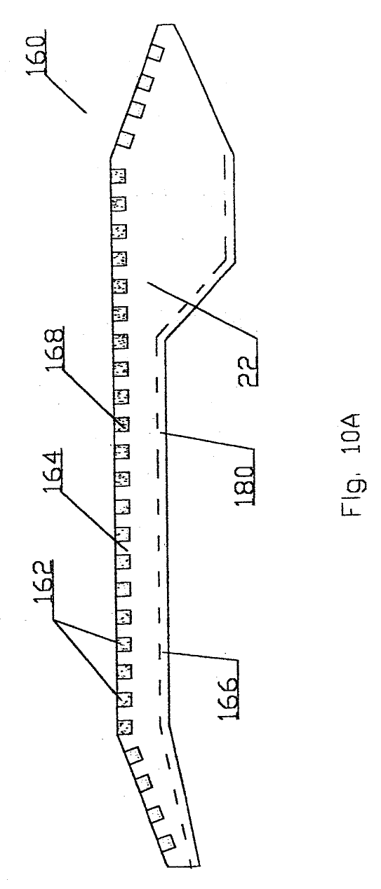

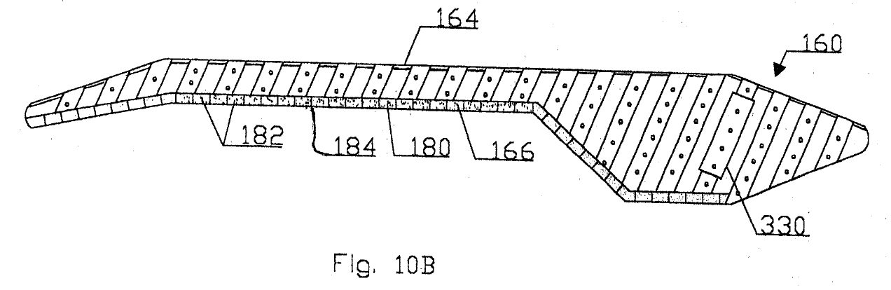

Another form of surgical cast venting device described in my earlier U.S. patent application Ser. No. 09/533,214 is illustrated in FIGS. 10A and 10B of the drawings. This venting device 160 is similar to the venting device 1, illustrated in FIGS. 1 and 2, except for the differences noted hereinafter. In particular, this venting device includes a multiple hook array fastener arrangement indicated generally at 162 that extends along and is attached to a first longitudinal edge section 164 of the elongate strip of porous fabric material 22. The arrangement 162 is adapted to detachably secure the longitudinal edge section 164 to an adjacent second longitudinal edge section indicated at 166 when the elongate strip of material is wound around the part of the human body that is to be enclosed by a cast. In fact, the second longitudinal edge section is the edge section extending along the opposite longitudinal edge of the strip. In the embodiment illustrated in FIG. 10A, the hook array fastener arrangement comprises a series of spaced apart, hook array fastener devices or pads 168 distributed along the first longitudinal edge section. Preferably these fastener devices are attached to the edge section by bonding using suitable adhesive or by being stitched thereto using a suitable, stretchable elastic thread. It will be understood that this elongate strip 22 is made with a fabric material which can be stretched to some extent as it is wound around the body part, thus allowing it to fit snugly. Although the hook array fastener devices or pads 168 may not themselves be stretchable, whether these devices are or are not spaced apart, for example by the length of the pads themselves and by attaching the fastener devices using elastic thread, the ability of the elongate strip 22 to stretch during use is not substantially or detrimentally affected.

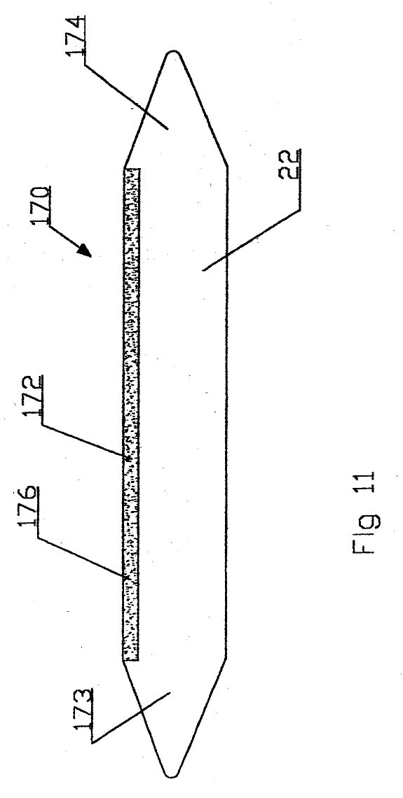

In the alternative version of the venting device illustrated in FIG. 11, the venting device 170 is the same as that shown in FIGS. 10A and 10B except that the hook array fastener arrangement extending along the upper longitudinal edge comprises an elongate hook array fastener strip 172 that extends at least a substantial portion of the length of the elongate strip of fabric material. As illustrated, the fastener strip 172 extends the entire length of the venting device except for the tapered end sections at 173 and 174. In this version, the fastener strip 172 is preferably made from a stretchable, elastomeric strip of material so that it can stretch with the preferred gauze material forming the strip 22. Fastener strip 172 can be secured by stitching made with elastic threads indicated at 176 to the elongate strip of fabric material. For purposes of the present application, it will be understood that the terms “hook array fastener arrangement” and “hook array fastener strip” as used herein refer to the use of well known and commonly used hook-type fasteners that are used in a variety of other products where fasteners are required. This type of fastener strip is commonly sold under the trademark VELCRO.

In the illustrated venting devices of FIGS. 10A, 10B and 11, there is also a multiple loop array fastener arrangement indicated generally at 180 that extends along the second longitudinal edge section 166 of the elongate strip and that is adapted for attaching to the hook array fastener arrangement 162 or 172. Again, it will be understood that the multiple loop array fastener arrangement can be constructed in a known manner using pads or strips formed with tough, multiple loops on their outer surfaces, which loops will engage readily with and hold on to the numerous small hooks distributed on the surface of the hook array fastener arrangement 162 or on the strip 172. Such loop array fastener arrangements are also commonly available and sold under the trademark VELCRO.

One form of loop array fastener arrangement is illustrated in FIG. 10B wherein there is shown a substantially continuous band of loop array fasteners extending along the second longitudinal edge section and secured thereto by stitching made from elastic threads (not shown but similar to the stitching 176 illustrated in FIG. 11). It will be understood that the illustrated band can be formed by either a continuous, stretchable strip forming the fastener arrangement or by a series of short loop array fastener strips or pads 182, again attached to the fabric by elastic stitching. These pads 182 are separated from one another by transverse joints 184. Where a series of separate pads are used along the second longitudinal edge, these pads or fastener devices need not themselves be made from a stretchable or elastomeric material, sufficient stretchability being provided by the numerous joints 184 and the stretchable or elastic thread used to attach the pads to the elongate fabric material. It will be appreciated that the provision of a substantially continuous band for the loop array fastener arrangement is preferred so that there is no difficulty in attaching the multiple hook array fastener arrangement 162 to the loop array fastener arrangement, for example, due to an inability to match up a particular hook array fastener device with a loop array fastener pad when the venting device has been wound around a body part.

In addition to or in the alternative to the use of elastic threads to attach the VELCRO, such as the pads 182, it is also possible and desirable to use a known type of V-shaped stitching that allows the stitching and the fabric to stretch during use. It is believed unnecessary to illustrate this type of stitch as it is commonly used in garments that must be able to stretch. This known stitching forms a zigzag pattern like the teeth on a standard sawblade.

It should be further appreciated that the use of a multiple loop array fastener arrangement along the second longitudinal edge section is not always required or preferred, the need for same being dependent upon the particular gauze or fabric material used to form the elongate strip 22. If, for example, the fabric material is sufficiently strong and porous so that it can be readily penetrated by the small hooks of the fastener arrangement at 162, then it will be possible generally to attach the longitudinal edge sections of the venting device together without the use of any loop array fastener arrangement. In such case, the second longitudinal edge section at 166 will simply be left bare on the inside surface so that it is available for securing to the hook array fastener devices. It will also be understood that although square rectangular fastener devices are illustrated in FIGS. 10A and 10B, these fastener devices can also have other configurations, for example, circular or elliptical and these fastener devices can be spaced closer together or further apart than illustrated in FIG. 10A.

Turning now to FIG. 12 of the drawings, this figure illustrates the possibility of connecting together several elongate fabric strips by means of “Velcro” type fastener arrangements. In the illustrated embodiment, there are two end sections or strips 190 and 192 and one straight middle section 194. Although only one middle section 194 is illustrated, it will be appreciated that there can be two or more similar middle sections depending upon the length of the final venting device required for the cast. Each of the three sections 190, 192 and 194 is provided with a hook array fastener arrangement 162 located along its upper longitudinal edge section and arranged on the outer surface. Extending along the inner ends 196 and 198 of the outer end sections are further multiple hook array fastener arrangements indicated at 200 and 202, these fasteners also being on the outer surface. FIG. 12 illustrates two alternate forms for these inner end fasteners. In the fastener arrangement 200, there is a substantially continuous band of multiple hook array fasteners comprising a series of fastener pad or devices 204 separated by joints 206. These fastener pads are preferably attached to the outer surface by means of stitching made of elastic threads and thus some transverse stretching at the inner end 196 is possible. In the fastener arrangement 202, a series of separated hook type fastener pads 208 are distributed along the inner end 198. Spaces 210 are provided between these fastener pads. Arranged on the inner surface of the middle section 194 and located at opposite ends are multiple loop array fastener arrangements 212 and 214. These fastener arrangements can again be either in the form of continuous, elastic fastener strips or they each can be a series of loop array fastener pads or devices. It will be understood that the fastener arrangement 202 can be detachably connected to the fastener arrangement 214 and likewise the hook type fastener arrangement 200 can be detachably connected to the loop type fastener arrangement 212 in order to connect the three sections together. Further, if more than one middle section 194 is to be provided in order to produce a continuous venting device strip, it will be appreciated that some of the middle sections 194 can be provided with multiple hook-array fastener arrangements at opposite ends or they can be provided with a multiple hook array fastener arrangement at one end and a multiple loop array fastener arrangement at the opposite end. The particular type of middle section 194 that is used or provided will be dependent upon the hook or loop type fastener arrangement to which it is to be connected at each end thereof.

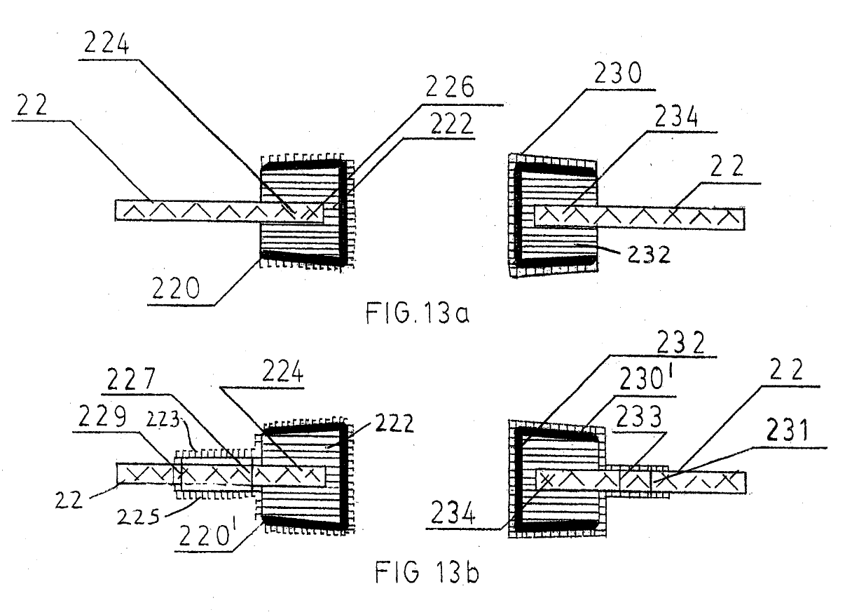

FIGS. 13a and 13b illustrate alternative ways of mounting hook or loop type fastener arrangements along the longitudinal edge or edge section of the elongate strip of porous fabric material or gauze. In the section of the fabric strip shown on the left side of FIG. 13a, there is a multiple hook array fastener arrangement 220 that is supported by an elongate, channel-shaped plastic edge support 222. The fastener arrangement 220 can be bonded by a suitable adhesive to the plastic edge support which in turn can be bounded by adhesive to a longitudinal edge section 224 of the fabric strip. This edge section can be inserted into the channel 226 prior to bonding taking place. It will be understood that the plastic edge support 222 together with the attached fastener arrangement 220 can either be in the form of a continuous, flexible strip extending most or all of the length of the fabric strip 22 or in the form of a series of short strips, for example, only having the length of the individual fastening devices extending along the edge section 164 shown in FIG. 10A. Similarly, a multiple loop array fastener arrangement 230 can be adhesively bonded to another channel-shaped plastic edge support 232 extending along the opposite longitudinal edge of the fabric strip 22. The second longitudinal edge section 234 is placed in the channel and is held therein by a suitable adhesive. Both the hook type fastener arrangement 220 and the loop type fastener arrangement 230 can extend around three sides of the adjacent plastic edge support as shown in FIG. 13a. With this arrangement, even if there is a slight overlap of the adjacent longitudinal edges, these longitudinal edges can still be securely connected together using the hook and loop type fastener arrangements.

Instead of using adhesive to bond the plastic edge supports to the fabric strip, it is also possible to mold these plastic strips in place on the longitudinal edges so that they are securely held on these edges by the molding process. The hook or loop type fastener arrangement can then be subsequently bonded to the plastic edge support after it has been molded and has hardened.

On the left side of the version illustrated in FIG. 13b there is a wider multiple hook array fastener arrangement 220′ that is supported by plastic edge support 222 and a longitudinal edge section of the strip 22. The fastener arrangement 220′ can again be bonded to the plastic edge support but the flat sections 223 and 225 can be attached to the fabric by a longitudinal line of stitching at 227. There can be a second line of stitching at 229. Similarly, on the right side of this version there is a wider multiple loop fastener arrangement 230′ that is adhesively bonded to the edge support 232 and is also connected by stitching at 233 to a longitudinal edge section of the strip 22. Additional stitching 231 at the inner edge of the fastener arrangement can also be provided.

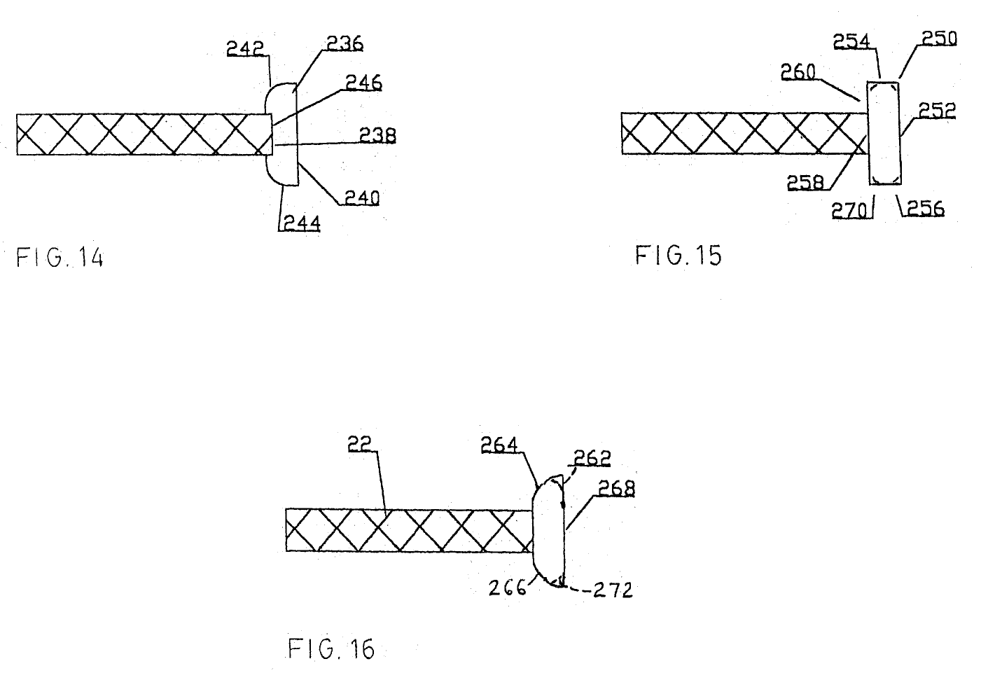

FIG. 14 to 16 illustrate alternative cross-sections for the plastic edge supports to which the hook or loop type fastener arrangement can be bonded. In the embodiment of FIG. 14, the edge support 236 has only a very shallow groove at 238 and, opposite this groove, is a flat outer side 240. Extending between the shallow groove and the side 240 are upper and lower curved sides 242 and 244. Again, adhesive can be used to bond the edge 246 to the plastic edge support. The edge support 236 could also be made without any shallow groove 238 (as per the versions of FIGS. 15 and 16 described below).

The plastic edge support 250 of FIG. 15 has a substantially rectangular cross section with a flat outer side 252 and no channel formed in the opposite side. The top and bottom sides 254 and 256 of the edge support are substantially narrower than the width of the outer side 252. With this edge support, the edge of the fabric strip at 258 is bonded directly to flat inner side 260. It is also possible to make this version 250 with an attachment groove in the side 260 similar to the groove 238.

The edge support 262 shown in FIG. 16 is somewhat similar to the edge support 250 as it has no channel to receive the edge of the fabric strip. However, with this edge support, there are upper and lower curved sides 264 and 266 that extend between the longitudinal edge of the fabric strip 22 and a flat, elongate outer side 268. It is also possible to make the support 262 with an attachment groove similar to the groove 238 where the curved sides 264 and 266 meet. In FIGS. 14 to 16, the hook or loop type fastener arrangement has been omitted but it will be understood that this arrangement is bonded to at least the outer side of the plastic edge support and optionally on the curved or flat outer and top surfaces as well. Also, instead of sharp corners or edges extending along the length of the plastic edge support, it is also possible to round the longitudinally extending edges as indicated by the dash lines 270 in FIG. 15 and the dash lines 272 in FIG. 16.

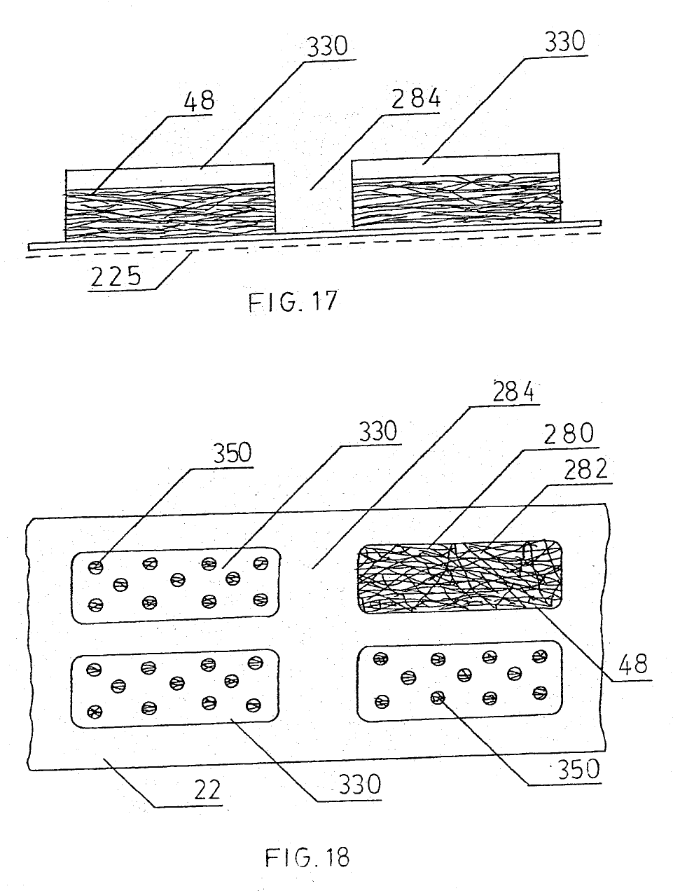

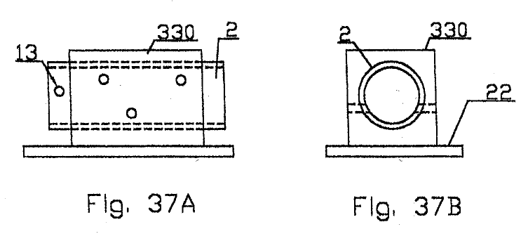

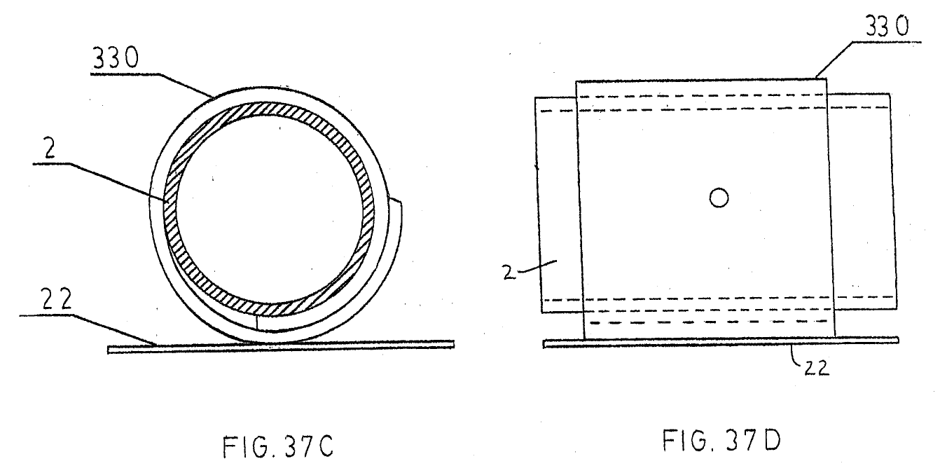

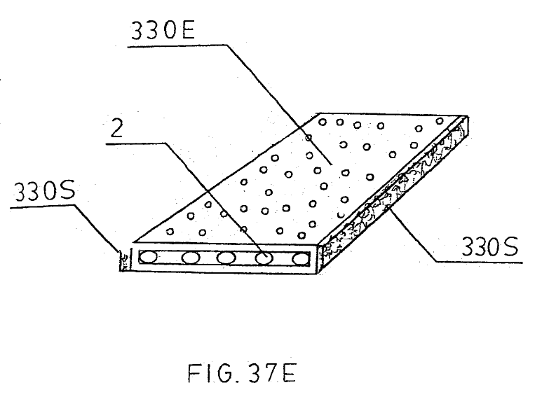

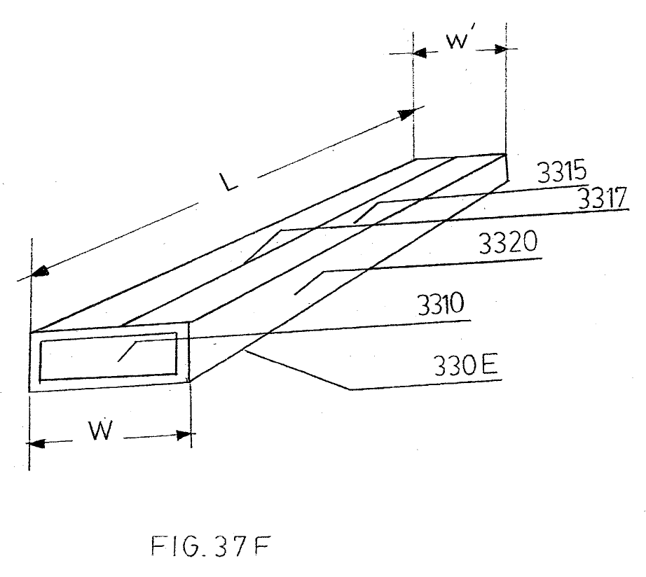

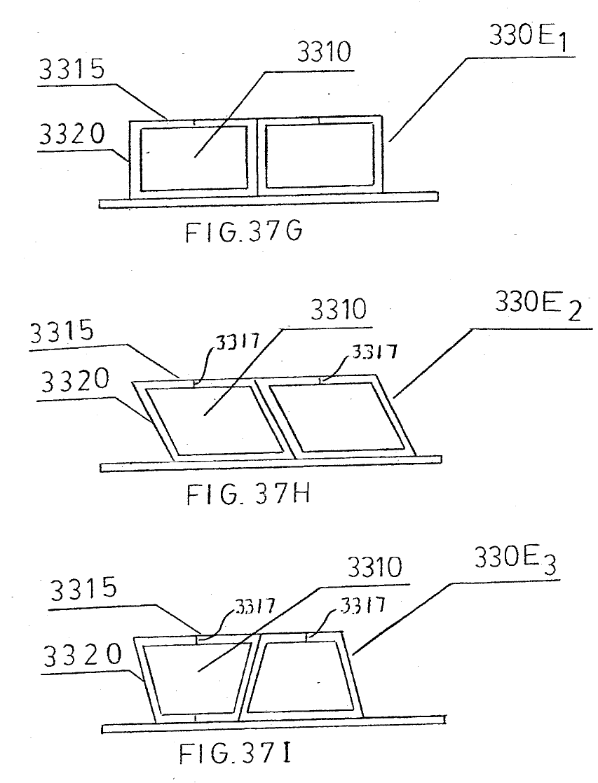

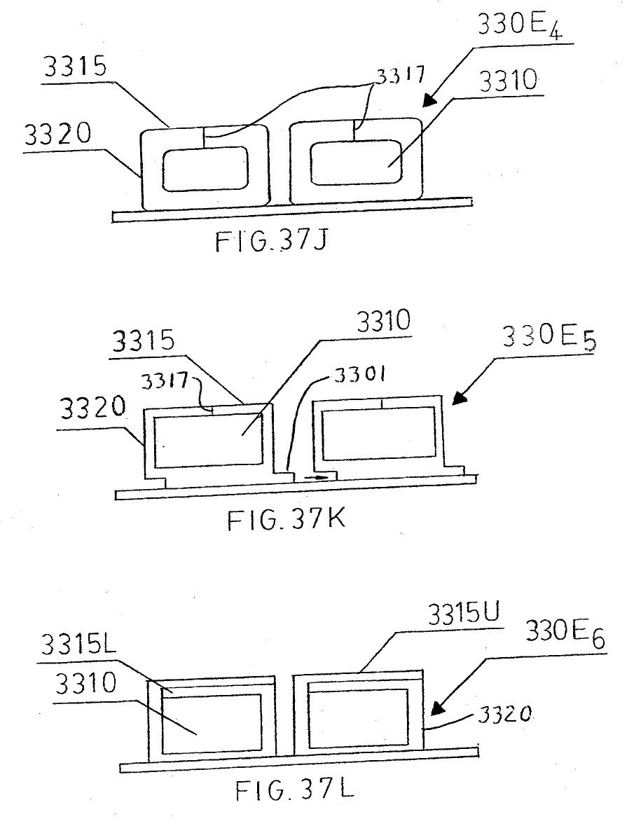

The plastic aerating devices affixed to and located on the inner surface of the venting device can take various forms other than the elongate plastic tubes illustrated in FIGS. 1 and 2. One alternate form of aerating device described in my pending U.S. patent application Ser. No. 09/533,214 is illustrated in FIGS. 17 and 18. These aerating devices are flexible layers 48 of interconnected, non-woven plastic threads or strips 280. Each layer has numerous small air passageways indicated at 282 that are formed therein. The thickness of each layer can be only one quarter inch or less and the layers can be separated by longitudinally and transversely extending air gaps 284. The layers are again adhesively bonded to a strip of fabric material 22 and it will be understood that the layers 48 are arranged on the inner surface of this material. The layers 48 have a consistency or makeup similar to natural “loofah” commonly used as a body sponge or body cleaning product. However, the layers 48 are made sufficient thin and they are provided with sufficiently large air spaces between the plastic threads that each layer is flexible and is able to bend to the body contour. Shown in FIGS. 17 and 18 is the attachment of padding 330 preferably EVA, with perforations 350 to aid in aeration. Alternately, padding may be in the form of padding envelopes as shown in FIGS. 37E to 37L, with aerating devices inserted therein.

Although the illustrated layers are rectangular in shape, other shapes are also possible, including square, triangular and elongate strips. Note that the layers may be utilized on any of the venting devices described herein. Also, the size of each layer and the size and number of air gaps between the layers can vary depending upon the flexibility of each layer and the proposed use for the particular venting device. Preferably the venting device employing these layers 48 is sufficiently stretchable to be permit it to be pulled snugly around the body part and the arrangement of the layers 48 must be sufficiently flexible as well to permit the venting device to adjust itself closely to the contour of the body prior to application of the cast material.

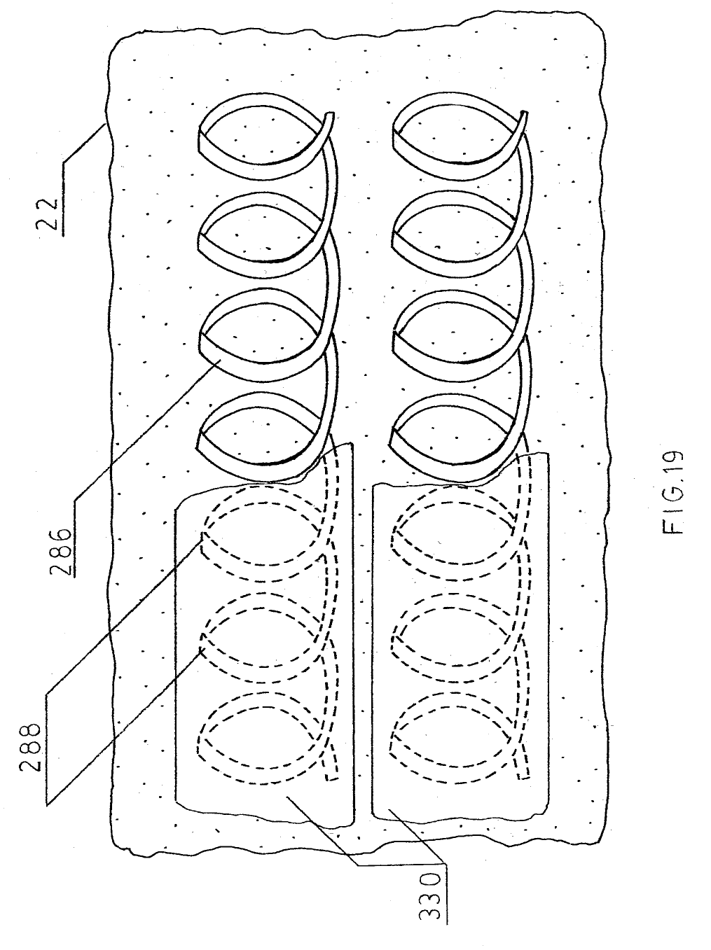

Another form of aerating devices is illustrated in FIG. 19 which shows only a portion of the inner surface of the venting device in the form of a fabric strip 22. In this version, the aerating devices comprise a substantial number of spiral-shaped, resilient plastic members 286 which are distributed over the inner surface of the venting device. The spiral shaped members can extend in a longitudinal direction of the venting device, but preferably extend transversely to the elongate strip of fabric. Each of the plastic members 286 is close to but spaced apart from adjacent ones of these members 286. The members preferably have a maximum exterior diameter less than one half inch and preferably no more than one quarter inch. The thread or wire from which member 286 is formed should have sufficient diameter or thickness that it will not irritate or cut a patient's skin when the venting device is put in place. Furthermore, adjacent individual spirals 288 should be reasonably closely spaced, again so they will not irritate or cut into the patient's skin, which may be sensitive and also so that the member 286 can adequately support the surrounding rigid cast without collapsing under the applied force. Padding 330, preferably EVA may be affixed over a portion of the members 286 or over the entirety of some or all of the members 286. Alternately, padding may be in the form of padding envelopes as shown in FIGS. 37E to 37L, with groups of members 290 inserted therein.

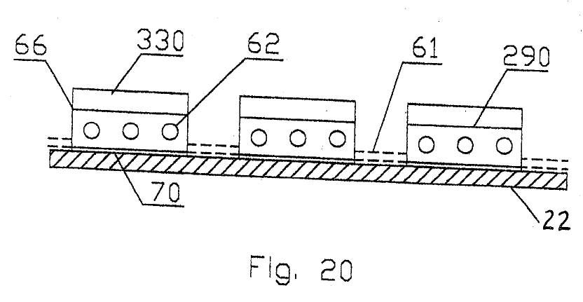

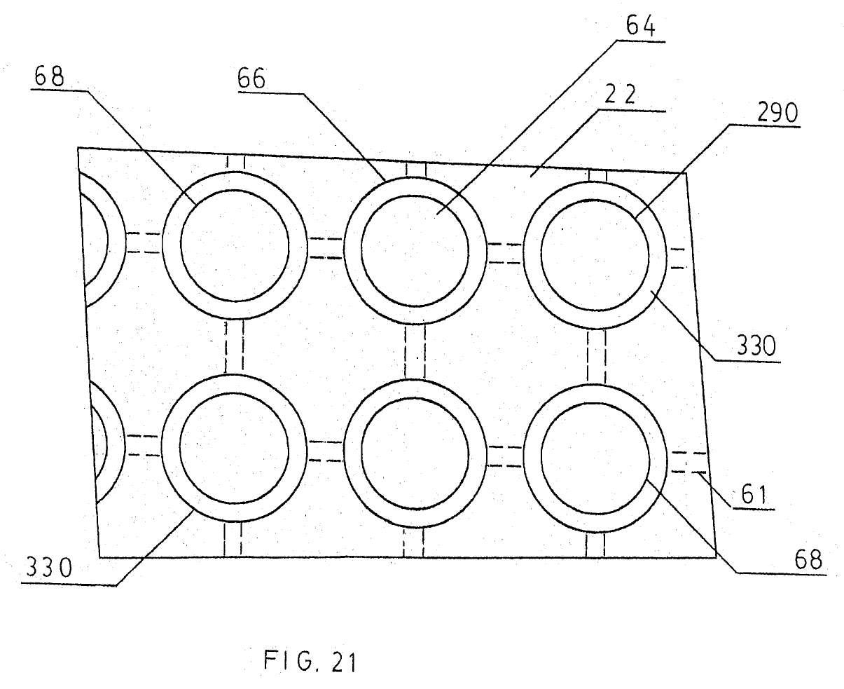

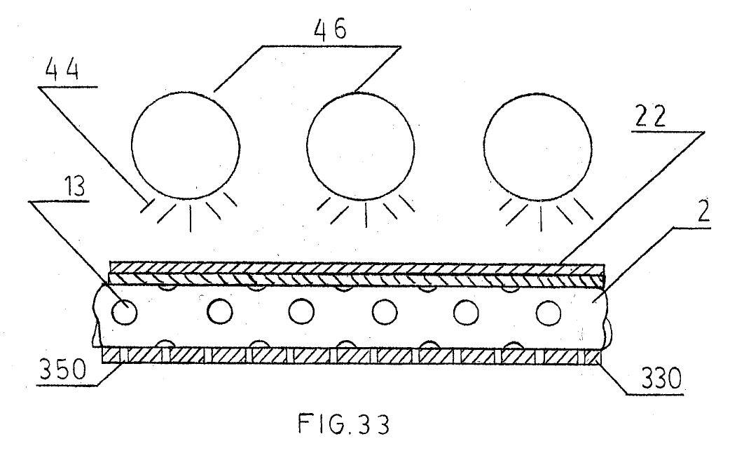

Turning now to a further embodiment of venting device illustrated in my pending U.S. patent application, this embodiment being illustrated in FIGS. 20 and 21, the plastic members which are affixed to the fabric strip 22 are in the form of ring-like plastic members 290, each of which is perforated with a number of small holes 62 about its perimeter. These holes permit the passage of air to and from a circular space 64 located inside each ring-like member. The plastic members 290 are distributed evenly or substantially uniformly over the inner surface of the fabric strip. The size of the member 290 can vary and will depend to some extent on the particular use to which the venting device is to be put. Typically, the preferred exterior diameter of each ring-like member is no more than one inch and more preferably is no more than one half inch. The thickness or depth of each ring-like member can also vary but preferably is no more than one quarter inch. The outer, annular edges of each member 290, these edges being indicated at 66, can be rounded if desired to avoid unnecessary irritation of the user's skin and to make the wearing of the venting device as comfortable as possible. The annular interior edges 68 can also be rounded, if desired. The members 290 are preferably bonded by a suitable medical adhesive indicated at 70. If desired, the ring-like members 290 can be attached to the fabric material or gauze in a manner similar to the elongate tubular members, this procedure being described hereinafter in conjunction with FIG. 33 of the drawings. Padding 330, preferably EVA, is affixed to some or all of the ring like members 290. This padding may be cut to match the size of the annular tops of the members 290. Alternately, padding may be cut such that padding is of larger diameter than that of the ring like members and thus the padding extends beyond the outer edges of the ring like members. Alternately, padding may be in the form of padding envelopes as shown in FIGS. 37E to 37L, with groups of members 290 inserted therein.

Optionally, the ring-like members 290 can be interconnected by integral plastic web connectors 61 illustrated in dashed lines in FIGS. 20 and 21. These web connectors can interconnect adjacent members 290 in order to maintain the uniform spacing of the members, particularly before they are bonded to the fabric. The plastic connectors 61 help support the ring-like members 290 even after the latter have been bonded to the fabric and help to prevent the ring-like members from becoming detached or from becoming displaced from their desired position. If desired, these thin plastic connectors can be broken in order to provide the venting device with sufficient flexibility and stretchability when it is being applied to the body part.

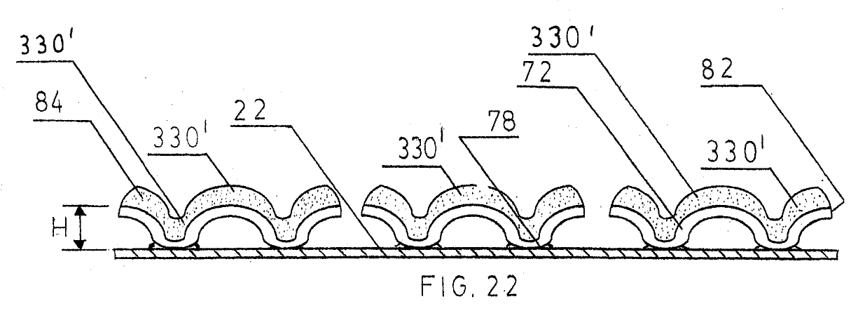

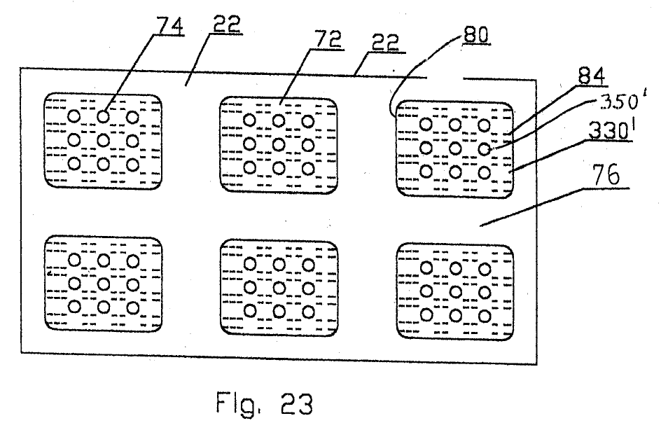

FIGS. 22 and 23 illustrate another modified version of a venting device described in my pending U.S. application Ser. No. 09/533,214, this version using aerating devices that are affixed to the inner surface of the fabric strip 22 and that are in the form of at least several and preferably a substantial number of corrugated plastic members 72, each of which is perforated with a substantial number of small holes 74. These holes can be circular and can be as small as 1/16th inch or less in diameter. These holes tend to increase the circulation of air about the corrugated members. The members 72 are distributed preferably uniformly over the inner surface of the fabric strip 22. Also, these members are preferably separated by longitudinally and transversely extending air gaps 76, the width of which can vary but preferably these gaps should be sufficiently wide to allow adequate air flow between the corrugated members and they should not be so wide as to allow contact between the rigid cast material and the patient's skin. Preferably the width of the gap is less than ½ inch while the preferred height H of each corrugated member 72 is no more than ¼ inch.

The members 72 are preferably affixed by a suitable medical adhesive indicated at 78. Preferably the opposite edges 80 and 82 are rounded to avoid irritation to adjacent skin and the edge region 84 forming each of the edges 80 and 82 should not extend towards the skin but should either extend parallel to the adjacent skin as shown or extend away from the skin. Although opposite edges 80, 82 are shown at a position H, the edges could be positioned at a lower point in the corrugation. Although only a couple of corrugations are shown on the illustrated plastic member 72, it will be appreciated that these members could have more corrugations such as four or five or more, particularly if the corrugations are made quite small in width. Padding 330′, preferably EVA, is affixed to the members 72 and its perforations 350′ may be aligned with the holes 74 in the members 72 to aid in aeration. Alternately, padding may be in the form of padding envelopes as shown in FIGS. 37E to 37L, with groups of members 72 devices inserted therein.

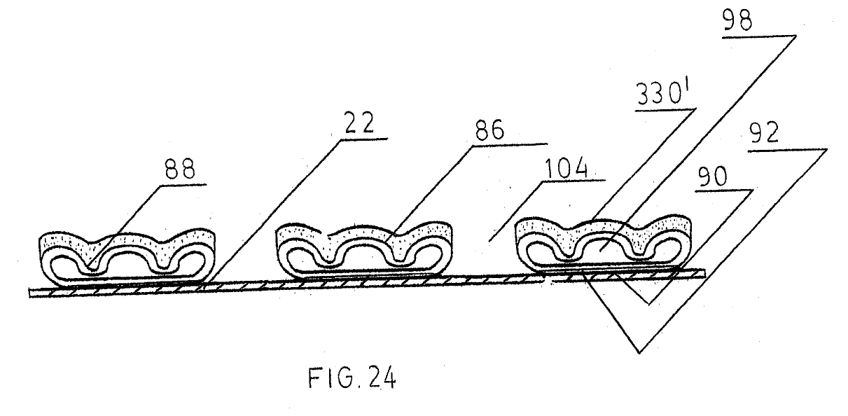

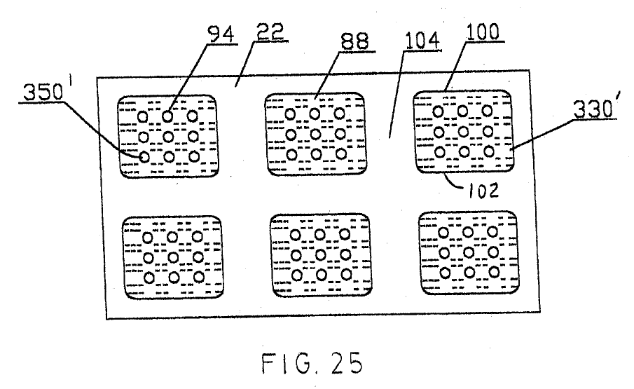

Still another type of aerating device for a venting device is illustrated in FIGS. 24 and 25. These aerating devices which are affixed to the inner surface of the fabric strip 22 are also corrugated plastic members but these members 86 have corrugated top sections 88 and planar bottoms 90 integrally connected to the top sections. The bottoms 90 are affixed by means of a suitable adhesive 92 to the porous fabric material or gauze. The top sections 88 are perforated with a substantial number of small holes 94 which can be round. These holes open into an air space 98 formed between each top section and its respective bottom 90. It will be understood that each corrugated member 86 is preferably open-ended at 100 and 102.

The corrugated members 86 may be spaced apart by longitudinally and transversely extending air gaps 104 which are sufficiently wide to permit the free flow of air between the members 86 and which are not so wide as to allow contact between the surrounding rigid cast and the patient's skin. Preferably the maximum width of the air gaps 104 is less than ½ inch. The dimensions and height of each corrugated member 86 can be similar to the corrugated members illustrated in FIGS. 22 and 23. Again, padding 330′, preferably EVA, with perforations 350′, is affixed to said members and the perforations 350′ may align with the air holes 94 to aid in aeration. Alternately, padding may be in the form of padding envelopes as shown in FIGS. 37E to 37L, with groups of members 86 inserted therein.

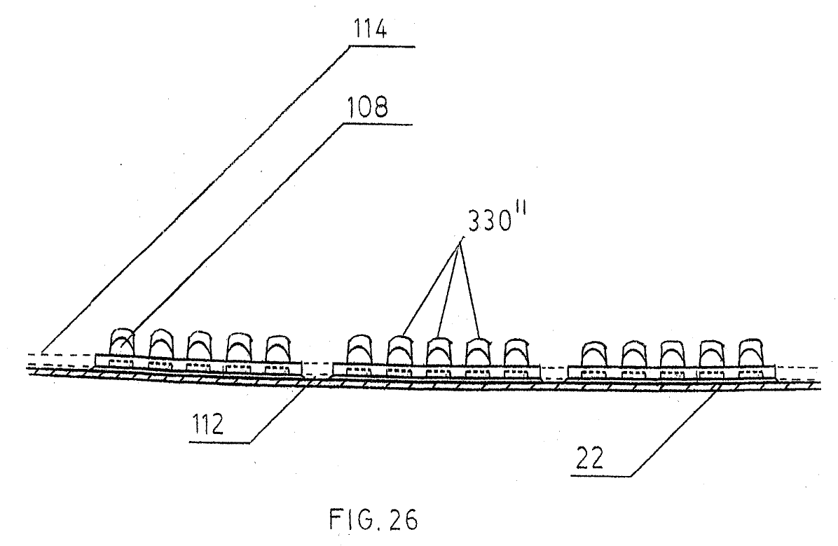

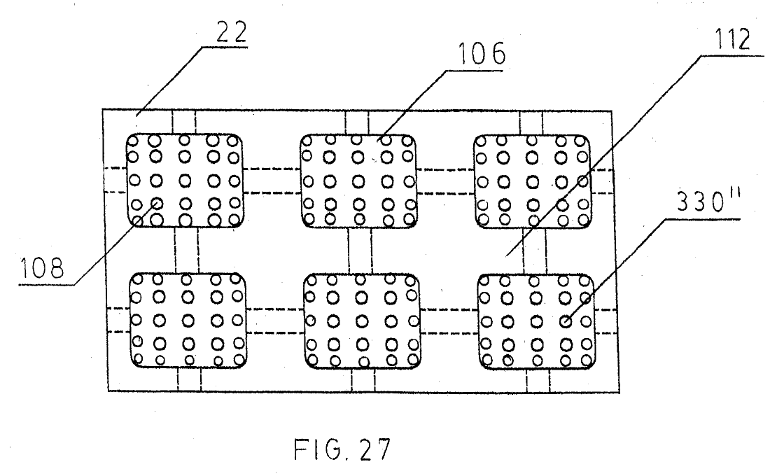

Yet another form of aerating device for a surgical cast venting device described in the aforementioned U.S. patent application is illustrated in FIGS. 26 and 27. In this embodiment, the aerating devices affixed to the inner surface of the fabric strip comprise at least several and preferably a substantial number of plastic members 106 with at least several and preferably a substantial number of bumps or protrubences 108 formed on an outer surface 110 of the plastic member. The bumps, which are preferably rounded as shown, project away from the inner surface of the fabric strip 22. The plastic members, which can have a square shape as shown, are preferably distributed evenly over the inner surface of the strip and are spaced apart from one another. There are longitudinally and transversely extending air gaps 112 extending between the plastic members. These gaps can be similar in width to the air gaps 76 and 104 of the embodiments illustrated in FIGS. 22 to 25. Although the illustrated members have a generally square shape, they can also have a rectangular, triangular, or rounded shape. Although the members have twenty-five bumps 108 laid out in a regular grid on the outer surface, the number of bumps can be fewer or more than the number shown. Padding 330″, preferably EVA, is affixed to the top of some or all of the bumps 108. Alternately, a layer of padding may cover the entirety of the upper surface of the plastic members, thereby blanketing the bumps with padding material. Alternately, padding may be in the form of padding envelopes as shown in FIGS. 37E to 37L, with members positioned therein.

Optionally, the plastic members 106 can be interconnected by means of thin, plastic connecting members 114 which can be readily broken, if required. The connectors 114 can be formed integrally with the plastic members 106 in the molding process. By using these connectors, the plastic members 106 will be correctly spaced and oriented prior to bonding and as they are bonded by adhesive to the fabric strip. Again, after the members have been bonded to the fabric material, the thin plastic connectors 114 can be readily broken, if necessary, to provide the venting device with the desired stretchability and flexibility for mounting on a body part.

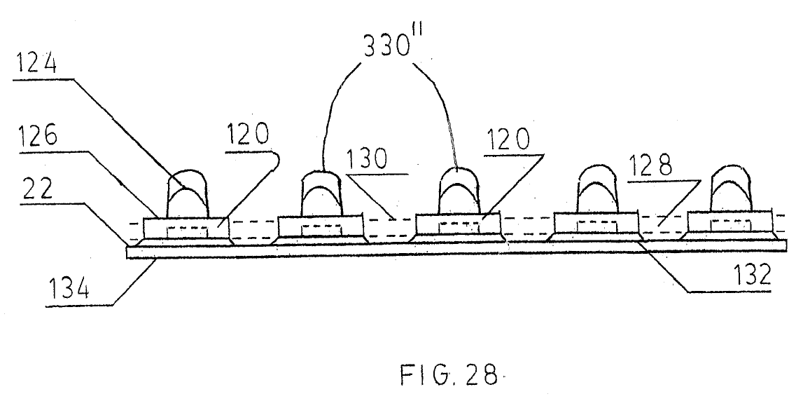

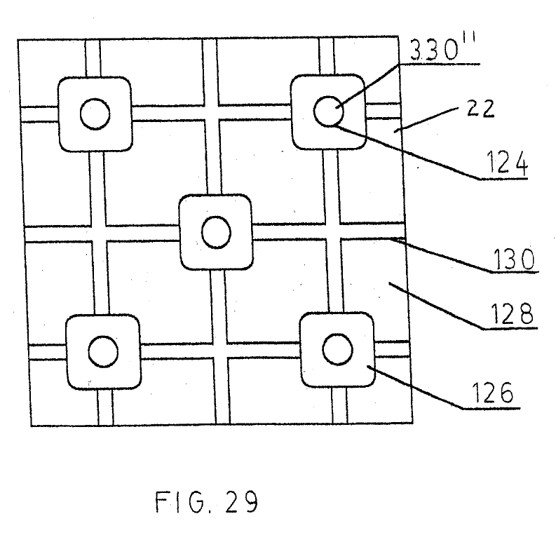

Yet another form of aerating device (which is also described in the aforementioned pending U.S. application) for use in a venting device is illustrated in FIGS. 28 and 29. In this version, the plastic aerating devices fixed to the inner surface of the fabric strip 22 are at least several and preferably a substantial number of plastic members 120 which can be quite small and relatively closely spaced. Unlike the members shown in FIGS. 26 and 27, each of the members 120 is formed with a single bump or protuberance 124 which is preferably rounded and centrally located on the outer surface 126. This single bump projects away from the inner surface of the fabric material. The plastic members 120 are uniformly distributed over the inner surface and are spaced apart from one another by longitudinally and transversely extending air gaps 128.

As in the previous embodiment, the members 120 can be optionally interconnected using integral connecting plastic webs or links 130 shown in dash lines in FIG. 28 but in solid lines in FIG. 29. These webs help to maintain the spacing in between the plastic members 120 and to maintain their orientation before they are bonded to the fabric material by adhesive. An adhesive 132 is applied to the outer surface 134 of the fabric and soaks through the fabric material to the adjacent surface of the plastic member 120 before being cured and dried. The web members 130 can readily be broken to give the venting device adequate flexibility and to permit the device to be stretched around the body part. Again, padding 330″, preferably EVA, may be attached to some or all of the bumps 124 or the members may be blanketed with a layer of padding. Alternately, padding may be in the form of padding envelopes as shown in FIGS. 37E to 37L, with members inserted therein.

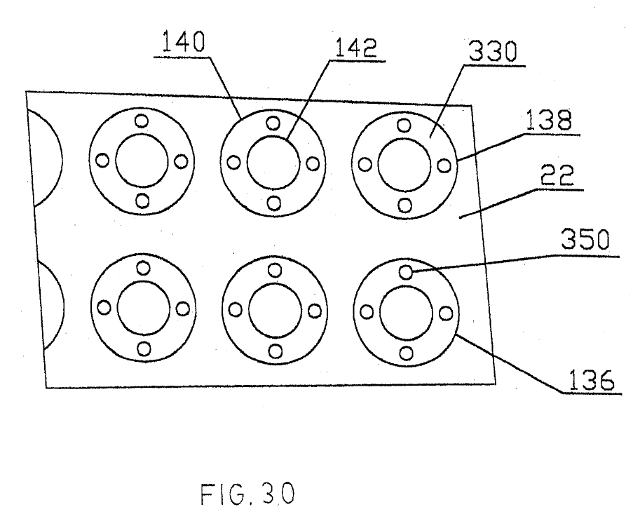

Another version of an aerating device described in the aforementioned pending U.S. application is illustrated in FIG. 30 wherein the aerating devices affixed to the inner surface of the strip 22 comprise a substantial number of ring-like plastic members 136 which are perforated with small holes 138 through the upper surface and/or side walls of the members. Although only four holes 138 are shown in each member in FIG. 30 for ease of illustration, it will be appreciated that the members 136 can have more small holes such as eight or more. The provision of the holes helps to reduce the area of the fabric material that is actually covered by the plastic of the ring members. The annular inner and outer edges 140, 142 of each ring-like member can be rounded to help lessen skin irritation by these members. The size of these members can be approximately the same size as the ring-like members 290 illustrated in FIGS. 20 and 21. Also, instead of being round members as illustrated, the members 136 could instead be oval-shaped or even square or rectangular in shape. Again, padding 330, preferably EVA is affixed to the outer surface of the members 136 and preferably at least some of the perforations 350 in the padding align with holes 138 in the members. Alternately, padding may be in the form of padding envelopes as shown in FIGS. 37E to 37L, with members 136 inserted therein.

Another form of aerating device similar to the plastic tubular members first described is illustrated in FIG. 31. In this version, there are again elongate tubular members but these plastic members 150 are somewhat flattened rather than being round in cross-section. In other words, these members 150 have an oval-shaped cross-section and because they can then be larger in size, they can have more small holes 152 formed in their sides. Otherwise the construction of this version of the venting device is substantially the same as the earlier described version such as that shown in FIGS. 1 and 2. Again, the plastic members 150 extend parallel to one another and are spaced apart generally a uniform distance. They are affixed by a suitable medical adhesive indicated at 154 to the fabric strip 22. Again, padding 330, preferably EVA, is affixed to the upper surface of some or all of said members 150.

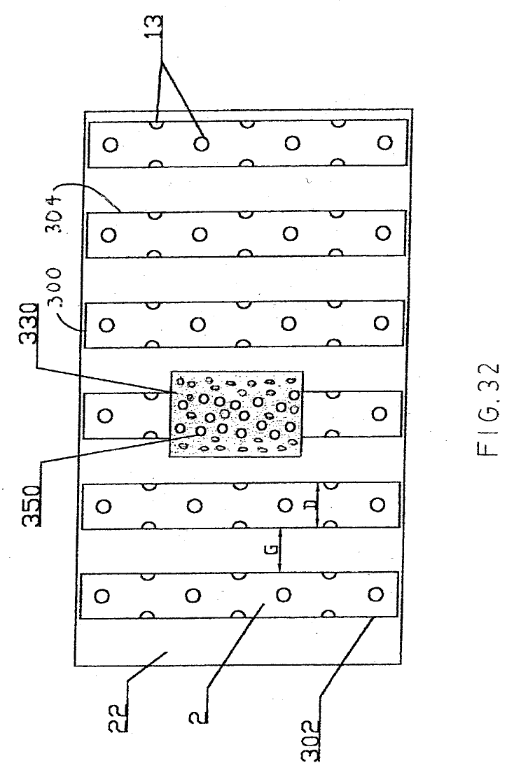

FIG. 32 illustrates some further details concerning the construction of a cast venting device which uses a substantial number of plastic tubular members 2 distributed over and affixed to the inner surface of the fabric strip 22. Preferably, each of these tubular members is open ended at both ends thereof including the end 300 illustrated in FIG. 32. For some applications of the venting strip and depending also on the nature of the plastic tubular member itself, the tubular member could be constructed with only one open end while still providing adequate ventilation under the cast.

Preferably the plastic tubular members 2 are elongate as illustrated in FIG. 32 and are adhesively bonded to the fabric material. The preferred adhesive is, as indicated, a non-toxic medical adhesive which is known in the medical art and which will bond to both the flexible plastic of the tubular members and the fabric material which is preferably cotton gauze.

The ventilation holes 13 can be round as shown and quite small, for example, 1/16 inch or less. There should be holes on the side of the tubular member opposite the fabric material and also on the two rounded sides indicated at 302 and 304 in FIG. 32 that are located at 90 degrees approximately to the aforementioned side. This arrangement enables air to pass freely into and out of the tubular members. Preferably the plastic from which the tubular members are made is a medical grade plastic and is selected so as to be as compatible with the skin as possible and non-allergenic. The plastic must also be sufficiently flexible to mold itself to the contour of the limb when the venting device is applied, particularly when the exterior cast material is being or has been applied about the venting device.

In one configuration, each plastic tubular member may be located close to but spaced apart from adjacent ones of the plastic tubular members as illustrated in FIG. 32. For example, the gap G indicated in FIG. 32 which may be substantially uniform can be approximately equal to the diameter D of the adjacent tubular members. A smaller relative gap or no gap may be preferred. The gap may be sufficient to allow adequate air flow in the longitudinal direction between the tubular members. Also, the gap G should not be too large in order that the tubular members can act as proper spacers to maintain the rigid cast material away from the adjacent skin area. In other words, the gap should not be so large that when the cast is applied, there is in effect no air gap in some locations between the skin and the rigid cast layer. Padding 330, preferably EVA, is affixed to portions of some or the entirety of all of said tubes 2, and perforations 350 in the padding preferably align with at least some of the holes 13 to aid in aeration. Positioning the tubes such that gap G is minimal may enhance comfort of the user by reducing the likelihood of pinching of the skin between tubes covered with padding 330. Although tubes are shown of a particular diameter, the diameter of any of the tubular members utilized with this invention and disclosed herein may be increased or decreased as desired by the user.

Another advantageous optional feature that can be employed in the above described venting devices is that of making the fabric material or layer 22 from thread or fibers having a distinctive color other than white or off-white. Preferably the fabric material is made with a bright color such as green or blue. By making the fabric material with a color that is distinctively different from the white plaster of paris material that forms the hard cast, a medical technician or doctor removing the cast by means of a saw or other cutting tool will easily know when the tool has passed through the hard cast material and has contacted the venting device. This will be revealed by colored fibers being thrown out or removed by the cutting device. The technician or doctor will then know that it is not necessary to cut any deeper in that region of the cast. This feature lessens the risk of the patient being inadvertently cut by the cutting tool.

FIG. 33 illustrates a preferred method for attaching either the plastic tubular members shown or other forms of aerating devices described herein to the strip of fabric material or the surgilast net. The individual tubular members 2 can be placed in a suitable holder of the required shape so that they can be held in their correct relative positions for the bonding process. With the tubular members correctly positioned, the fabric layer or strip 22 is placed over the holder and the tubular members and then a suitable medical adhesive is applied to the outer surface of the fabric along attachment areas directly opposite the tubular members or other aerating devices. Because of the porous nature of the fabric material or gauze, the adhesive soaks through the fabric to the tubular members at which point the adhesive is cured by exposure to ultraviolet light indicated schematically at 44 which can be created by ultraviolet light bulbs or tubes 46 arranged about the top surface of the fabric material. Padding 330, preferably EVA is attached to at least portions of said tubes 2 and perforations 350 align with at least some of the holes 13 in said tubes.

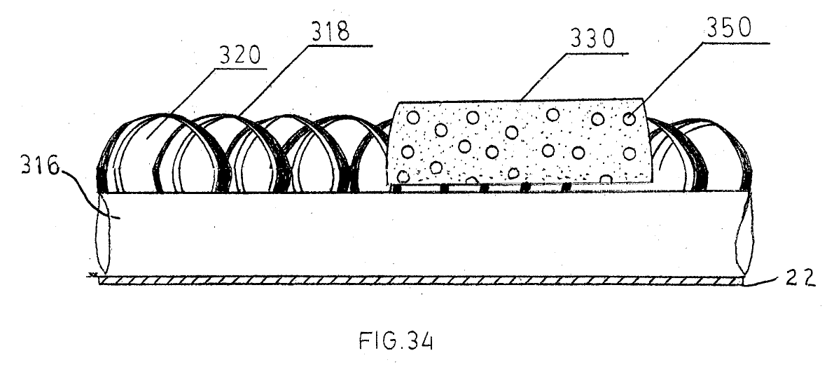

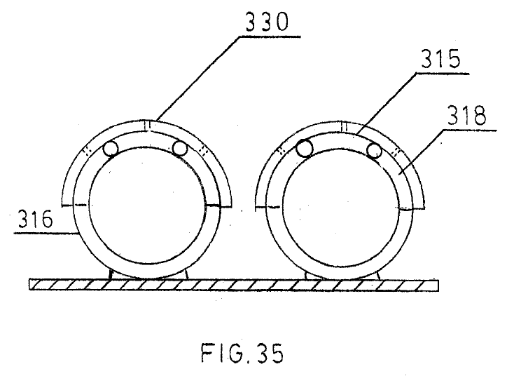

FIGS. 34 and 35 illustrate another possible form of plastic tubing member that is described in my aforementioned pending U.S. patent application and that can be used in a venting device. In this version each tubular member 315 has a substantially semi-cylindrical bottom half 316 which can be made of imperforate solid plastic or which can be perforated with circular holes (not shown) as per the embodiment shown in FIG. 32. However, the upper half (that is the half located furthest from the fabric strip 22) comprises a series of strong, durable plastic threads 318 that criss-cross one another as shown to form a semi-cylindrical arc that is sufficiently strong to maintain its shape during use of the venting strip. It will be appreciated that this arrangement leaves many holes or apertures 320 in the upper half through which air can pass and circulate. The plastic threads form an open “sieve-like” arrangement along the length of each tubular member 315. Padding 330, preferably EVA may be affixed upon the upper half of the tubing upon the threads and perforations 350 communicate with the many holes 320 on the upper half of the tube and thus air may pass therethrough, thereby maintaining the aeration capabilities of the device.

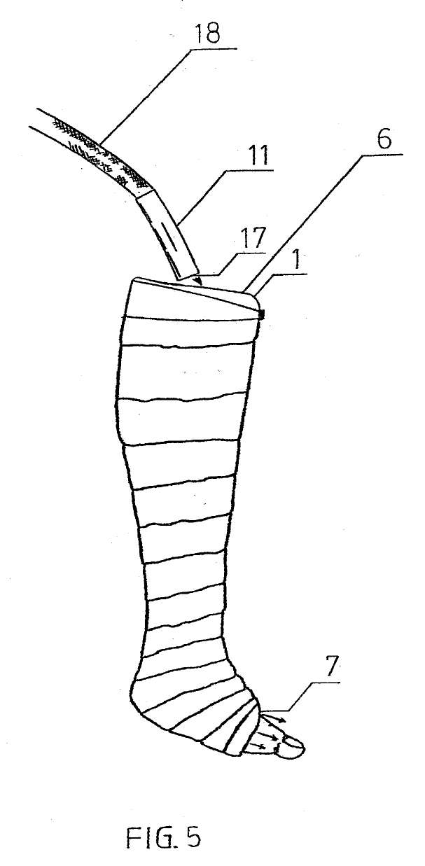

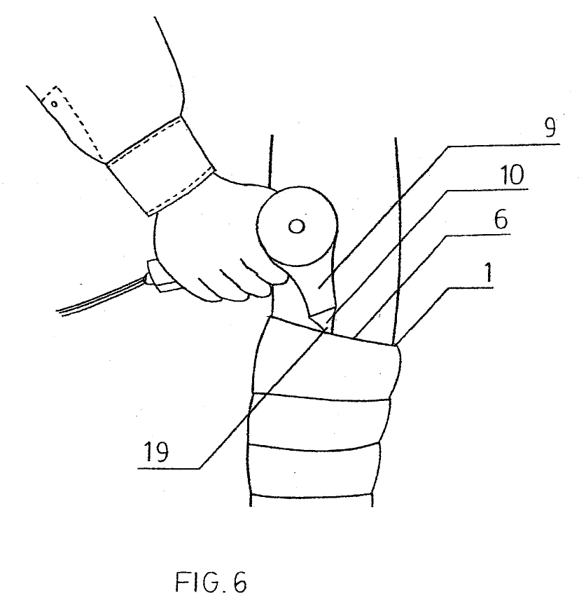

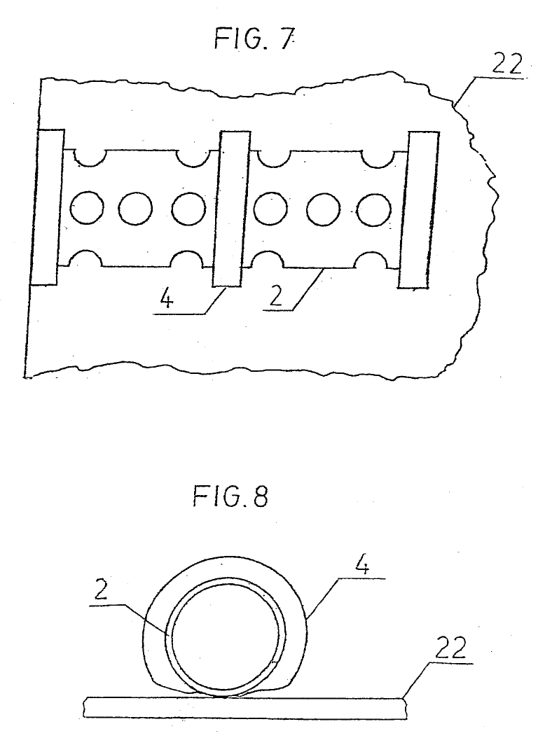

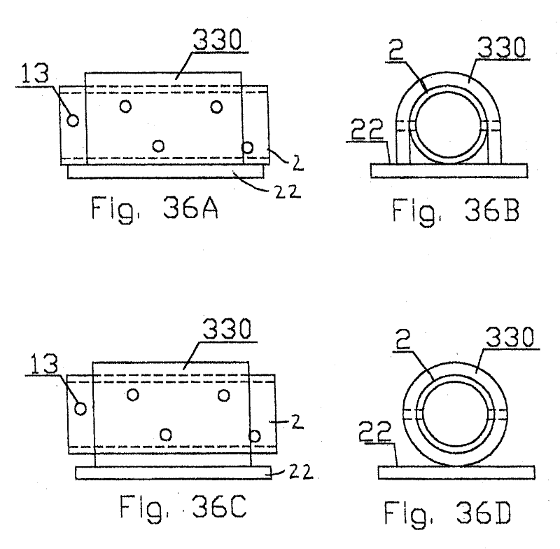

Although the figures above show specific embodiments of padding affixed to the aerating devices in FIGS. 1 to 35, various different shapes and embodiments of padding such as EVA may be used. For example shown in FIGS. 36A and 36B is an embodiment of padding attached around an inner longitudinal portion of a tube 2. The padding 330 is placed over the tube 2 such that the tube is adhered directly to the strip 22 and the padding is secured on the tube and is positioned to be disposed between the body part and the tube. Shown in FIGS. 36C and 36D is an embodiment wherein the padding 330 completely surrounds the tube 2 and the padding is adhered to the strip 22. FIG. 36D is an end view of the tube and padding illustrated in FIG. 36C. Shown in FIGS. 37A and 37B, is an embodiment wherein the padding 330 is a square shape in cross section and completely surrounds the tube 2 and adheres to the strip 22. FIG. 37B is a right end view of the padding and the tube shown in FIG. 37A. Shown in FIGS. 37C and 37D is an embodiment wherein the padding 330 is wrapped around the tube and overlaps itself for securement to the tube. Again, FIG. 37D is a right end view of the padding and tube illustrated in FIG. 37C.

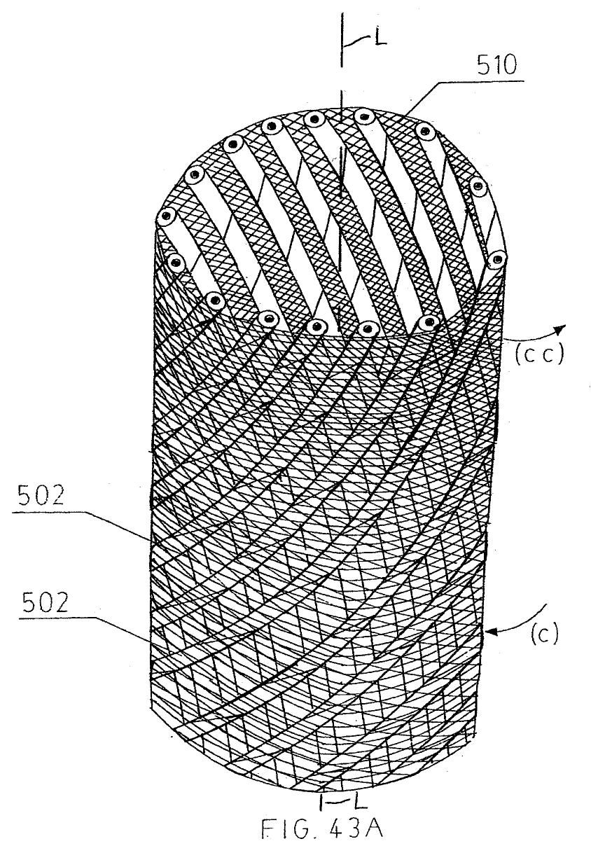

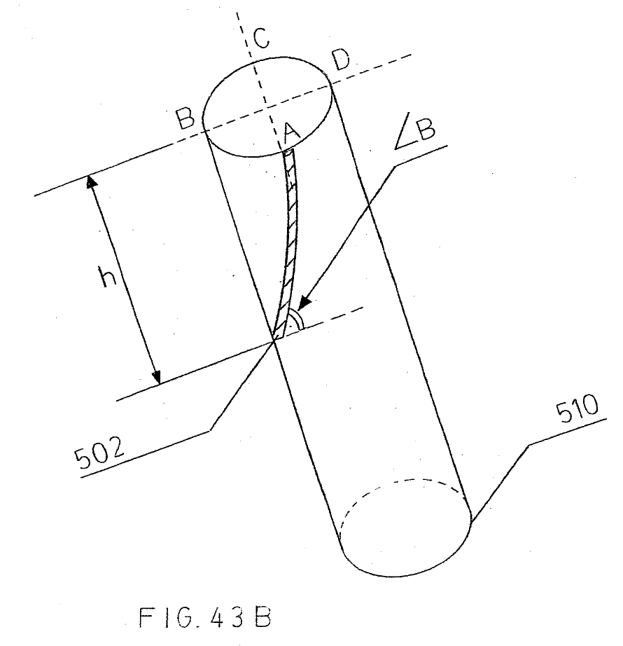

Shown in FIG. 37E is an embodiment wherein a plurality of tubes are wrapped within a padding envelope 330E. The preferred number of tubes wrapped within a padding envelope is in the range of one to six tubes. It will be understood that envelopes of this type with tubes therein can be wrapped about the limb in an elicoidal manner similar to that shown in FIG. 43A. Although FIG. 37E illustrates tubes positioned within the padding envelope 330E, it should be understood that any of the aerating devices described herein and disclosed in the figures may be positioned within the padding envelopes. In order to enhance comfort of the user, padding envelopes should be positioned on the venting devices such that no space exists between adjacent padding envelops. To further enhance comfort, a soft piece of padding 330S may be disposed between adjacent padding envelopes 330E. FIG. 37F to 37L illustrate further embodiments of padding envelopes into which any of the various aerating devices described and disclosed herein may be positioned. Aerating devices may be inserted through either open end (3310) of each padding envelope and adhered in place by any suitable means such as by means of application of medical adhesive. Alternately, an upper portion (3315) of the padding envelope may be removable from side portions (3320) or an upper portion may be secured only to one side portion (3320) thereby allowing the upper portion to be lifted open on the side not attached to the side portion. Alternately, the upper portion may include a slit (3317) separating the upper portion (3315) into two separate pieces, the slit (3317) extending across the upper portion such that the upper portion may be separated at the slit and aerating devices inserted into the envelopes through the slit.

Shown in FIG. 37L is an embodiment of a padding envelope (330E6) in which the upper portion 3315 includes an upper flap 3315U and a lower flap 3315L extending from side portions 3320 over the top of the envelope. Each flap is secured to one side portion only, the end of each flap in a friction fit with the other side portion. Therefore the flaps may be lifted open which allows aerating devices to be inserted into the envelopes. Aerating devices may be inserted into the envelope through the opening created when the upper flaps are lifted from the side portions. Once the aerating devices are inserted into the padding envelopes, the upper portion (3315) may be stapled or glued to the side portion or portions (3320).

Any number of padding envelopes may be secured upon the inner surface of any venting device disclosed herein with suitable medical adhesive. The padding envelopes are preferably positioned adjacent to each other, preferably without any gaps therebetween. Although each of FIGS. 37G to 37K show end views of two envelopes positioned adjacent to each other, a plurality of envelopes may be positioned adjacent to each other, preferably without any gaps therebetween. The absence of gaps therebetween reduces the likelihood of pinching and blistering of the skin between adjacent envelopes. In order to further ensure that no gaps occur between the envelopes and thereby further reduce the likelihood of pinching and blistering of skin, the embodiment of the padding envelope (330E5) shown in FIG. 37K illustrates portions 3301 of the padding envelopes overlapping so as to further ensure that no separation between the envelopes will occur. Where the venting strip is loosely wrapped around the body part, gaps may be present between the envelopes. To further enhance comfort of the user, a thin layer of material such as cotton, stockingnette, foam or Comfortem (TM) foam which preferably manages heat and perspiration may be adhered to the padding envelope, positioned between the limb and the padding envelop. In fact, such a thin layer of heat and perspiration absorbing material eliminates the need for the insertion of the preliminary layer of comparable such material by health care professionals thereby saving time and manpower.

The padding envelopes can be tapered along their length (L) and may be arranged in an elicoidal pattern on the inner surface of the venting strip. It is preferred that the padding envelope tapers along its length such that the width W of one open end 3310 of the envelope is greater than the width w′ of the other open end of the padding envelope. The padding envelopes may be arranged upon the venting strip such that they taper in a manner that follows the tapering of a typical human appendage such as an arm or a leg.

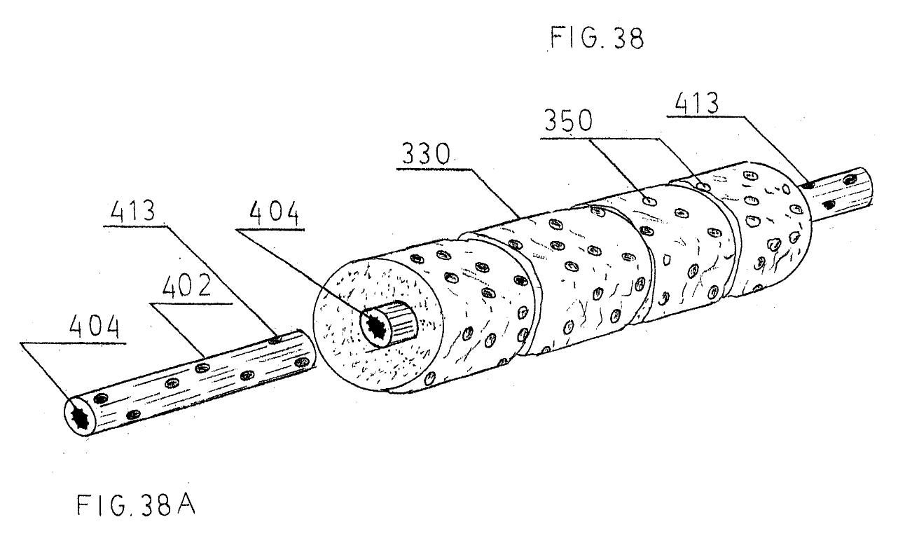

Shown in FIGS. 38 and 38A is an alternate tube 402 which can be used in an aerating device. The tube includes internal elongate ribbing 404 extending within the tube to prevent kinking of the tube. As seen in FIG. 38 the padding 330, preferably EVA with perforations 350, may be wrapped around this tube elicoidally (spirally). This elicoidally wrapped padding may include perforations 350 which preferably align with some perforations (holes) 413 in the tube in order to improve aeration.

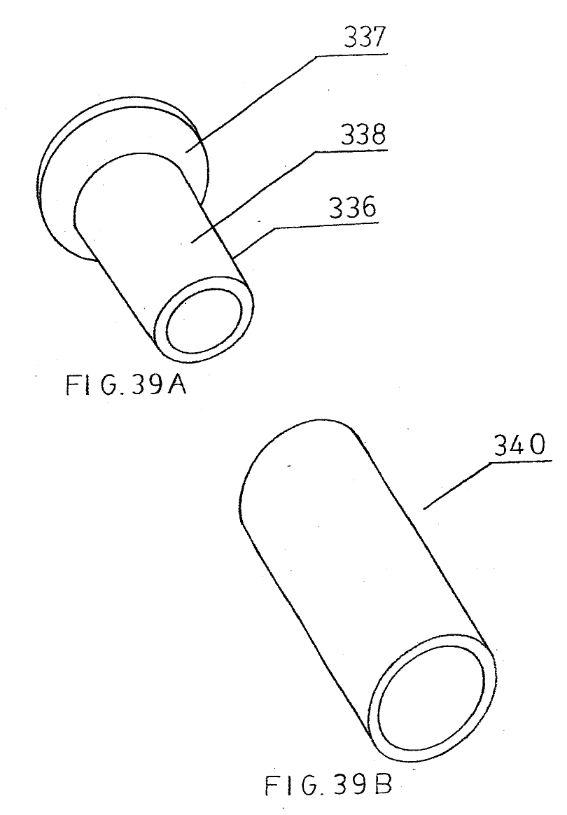

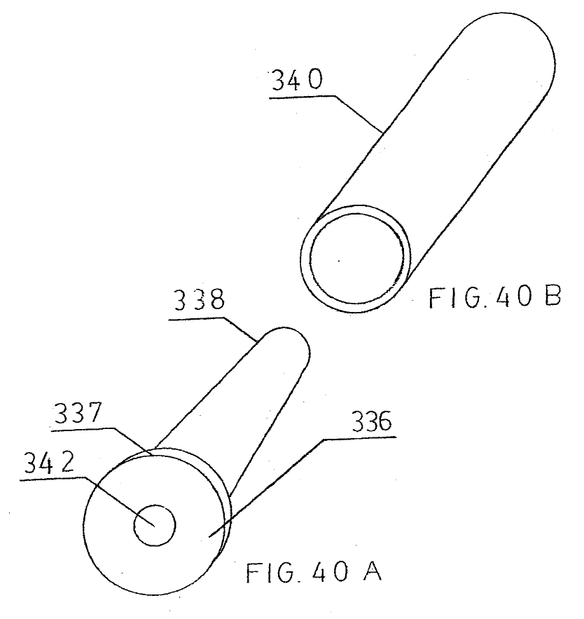

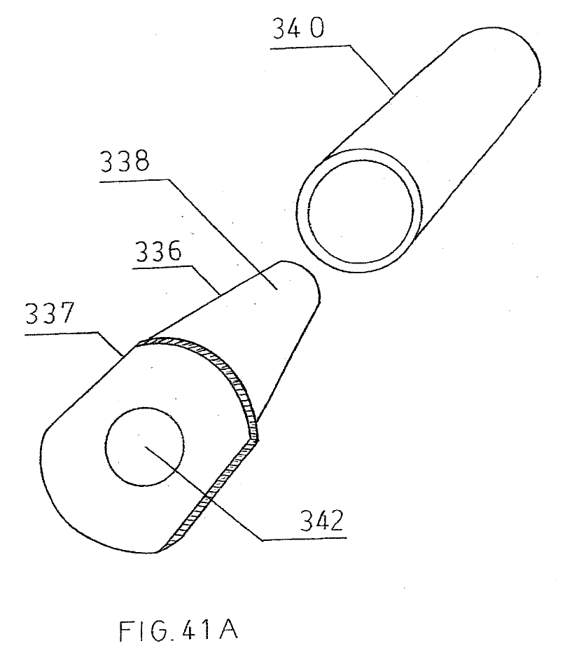

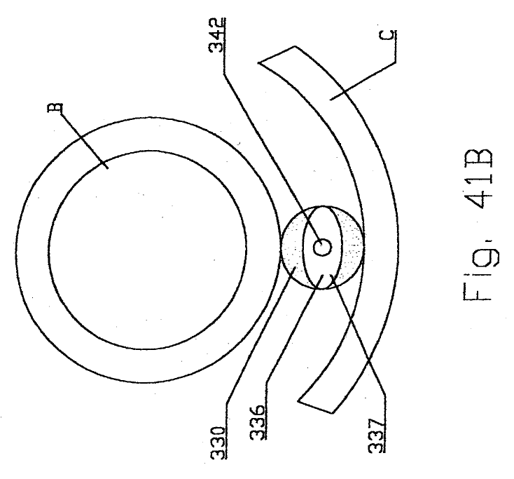

Shown in FIGS. 39A, 39B, 40A, 40B, 41A is a tube stopper 336 which will assist to fix in place the padding placed upon venting devices such as tubes. The tube stopper which can be formed with an end flange 337 and a tapered main section 338 is placed in an end of a tube 340. Shown in FIG. 41A is tube stopper having an alternate shaped end flange 337. The tube stoppers' end flange 337 helps to hold the EVA padding 330 in place on the tube. As shown in FIG. 41B, the tube stopper 336 also acts to absorbs some of the pressure caused by the appendage of the body B against the cast C where the tube stopper deforms as weight bears against it. A preferred form of the tube stopper has an end opening in which can be mounted an air filter 342 to help keep dust out of the tube.

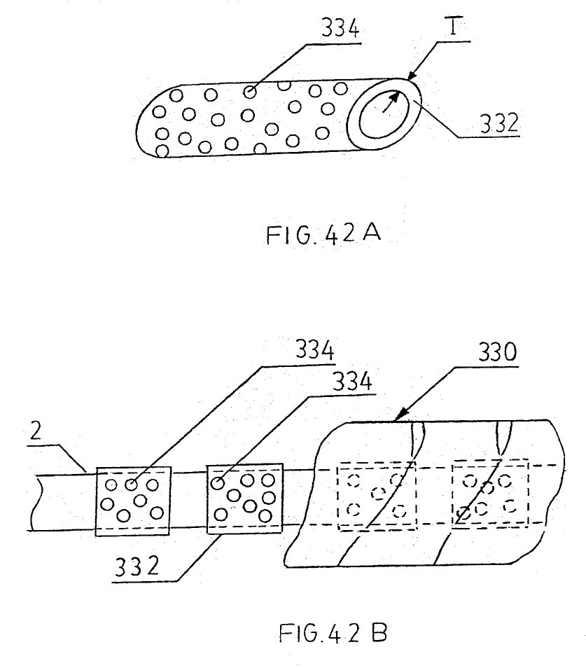

As shown in FIGS. 42A, 42B, in areas where the cast and venting device (which utilizes plastic tubular members 2 as aeration devices) bear a significant amount of weight from the appendage of the user, a plurality of ring like shaped cylinders 332 (preferably made of plastic, metal or metal threads) can be slid over and inserted on the tube. Preferably these cylinders will include perforations 334. These cylinders will add additional support for the tube and prevent flattening of the tubes. Padding material, such as EVA, 330 may be included over the cylinders and perforations 334 may align with perforations in the padding. In one preferred version of the cylinders 332, each cylinder has a length of about 1.5 cm and a cylindrical wall thickness T of 1.0 mm, but measurements may vary.

As shown in FIGS. 43A and 43B, tubes 502 in a venting apparatus can be arranged in an elicoidal orientation. The venting device in such an elicoidal arrangement can be applied to various human appendages and may include padding material 330, preferably EVA with perforations, placed over each tube 502 in the manner described above. Such a venting device may be disposed between an appendage and a cast, a brace, a support or immobilizer or back support or any similar device. This elicoidal arrangement of tubes may be used with the Surgilast net 510, which is a stretchable piece of fabric material having a significant amount of elasticity which allows the device to be slid over the desired body part. The padding such as EVA 330 may be applied elicoidally as in FIG. 38 or can be applied in any manner shown in the figures and described herein.

With reference to FIG. 43B, the height h through which a section of each plastic aerating tube extends is chosen so as to allow the Surgilast net on which the tubes are mounted to expand easily when the appendage or body part is being inserted into this venting device. This height “h” is proportional to the indicated acute angle B. Although the tubes 510 shown in FIGS. 43A and 43B are spaced apart, the device may be twisted about its longitudinal axis (L) such that the gap between the tubes 510 is increased or decreased.

The advantages of using the Surgilast net venting device shown in FIGS. 43A and 43B include the following:

(A) This tubular venting device can be twisted about axis L either clockwise or counterclockwise so as to change the distance between the padded tubes.

(B) Although illustrated using tubes, this tubular venting device may utilize any other aerating device described herein.

(C) This tubular venting device can open up or expand quickly and easily for placement over the injured body part or appendage.

(D) This expandable venting device adapts easily to fit over and around a complex body shape such as a foot.

(E) The device can be applied about a wide variety of body parts including an arm, an elbow, a wrist, and a leg or leg section, such as the thigh, knee, calf, foot or ankle.

(F) A suitably sized venting device of this type can be used around the torso to provide ventilation under either a body cast or under a back support.

The Surgilast venting device or any other tubular type venting device such as those disclosed in FIGS. 43A, 45 and 46 for example) can readily be placed over an injured appendage by using a suitable cardboard (or plastic) open-ended cone device with the venting device mounted thereon and applying it over the injured area (such as a leg) with the wider open end located towards the outer extremity of the appendage (i.e. the foot in the case of the leg). The cone may be round or oval in cross section or any other limb accepting shape. The cone device is then carefully withdrawn from inside the venting device so as to leave the latter in place and extending about the injured body part. It has been found that the open ended cone may tend to stick against the layer of padding such as EVA (which may be in the form of padding envelopes as discussed above) when being withdrawn from the between the venting device and body appendage. It has been found that when the layer of perspiration and heat absorbing material such as cotton stockingnette or Comfortem (TM) foam is disposed between the cone and the padding, the cone more effectively slides out with less likelihood of sticking, thereby leaving the heat and perspiration layer disposed between the skin and the venting device.

The elicoidally arranged tubes may be used inside boots and the like. However, the plastic tubing is not present beneath the foot if this venting device is used in conjunction with a walking boot or a walking aerated cast for a foot.

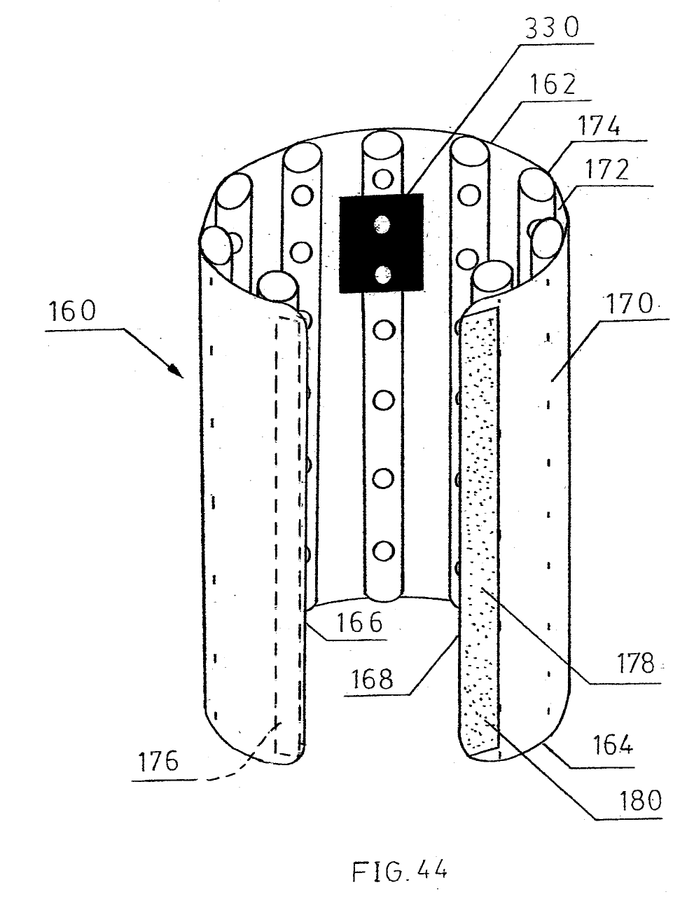

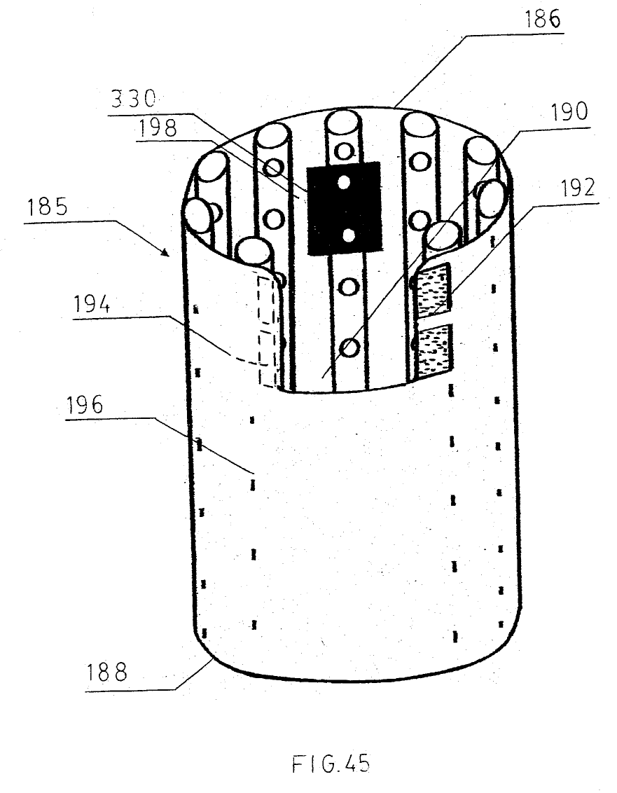

Padding can be applied on all venting devices described above and all venting devices disclosed in my pending U.S. patent application Ser. No. 09/476,106 filed Jan. 3, 2000, said application being incorporated herein by reference. FIGS. 44, 45 and 46 show embodiments from said application with padding 330 added. Shown in FIG. 44 is an embodiment indicated generally at 160, this device including a stretchable piece of fabric material having at least some elasticity and this stretchable piece has two opposite ends 162 and 164 and two opposite side edges 166 and 168. The stretchable piece also has an outer surface 170 and an inner surface 172. Again, aerating devices, preferably in the form of elongate plastic tubes 174 are affixed to and located on the inner surface 172 and can extend from the one end 162 to the opposite end 164.

Hook and loop type flexible fastener strips 176 and 178 are affixed to the two opposite side edges of this venting device and arranged for detachable connection to one another in order to form the piece of fabric material into a tubular configuration similar to the tubular configuration of the first embodiment. Alternatively, the venting device 160 can be detachably connected by means of the fastener strips to one or more similar venting devices to form a larger, tubular venting device, for example, one that can be applied around a patient's stomach region or chest region. In any event, it will be understood that this venting device is adapted for placement around part of the human body or animal body prior to application of a surgical cast over this part of the body. The fastener strips 176, 178 can be of standard construction and can be the type sold under the trademark or trade name Velcro. Thus, one of the strips, preferably the strip 178 on the outer surface of the fabric material, is covered with numerous small hook members 180 while the other strip, preferably the inside strip 176 outlined in dash lines in FIG. 44, is covered with numerous small loops made from strong threads or threadlike material. The fastener strips can be affixed in any suitable manner to the fabric material which is may be stockinet, surgilast, gauze, EVA or PLASTAZOTE or any like material. A preferred form of attachment for each of the fastener strips is a suitable, known form of medical adhesive but stitching or stapling can also be used to attach these strips.

As in the previously described venting devices, in the venting device 160 of FIG. 44, the aerating devices are tubular members 174 that are adhesively bonded to the fabric material on one side thereof. These aerating devices 174 cover at least a major portion of the inner surface in a substantially uniform manner in the preferred embodiment. It will be understood that the padding 330 can extend over all of the tubular members 174 and not just the small section as shown.

Yet another form of venting device is illustrated in FIG. 45, this device being indicated generally at 185 to which padding is added. The venting device 185 is substantially tubular, at least for a substantial portion of its length. The device is open-ended at 186 and 188. However, extending longitudinally from one end, in this case the end 186, is an elongate slot or gap 190. The purpose of this gap is to make it easier to pull or draw this venting device over a limb or other body part by enabling the end 186 to be opened up to some extent. After this venting device has been put in place and arranged on the body part, the gap 190 can then be closed by means of hook and loop type flexible fastener strips indicated at 192 and 194. The strips 192 on the outer surface 196 of the venting device are preferably strips with hook like members distributed over the exposed or outer surface. Preferably the fastener strips 194 that are located on the inner surface 198 of the venting device are covered with numerous small loops in a well known manner. It will be appreciated that the gap 190 can thus be closed by simply connecting the loop strips 194 to the hook type fastener strips 192.

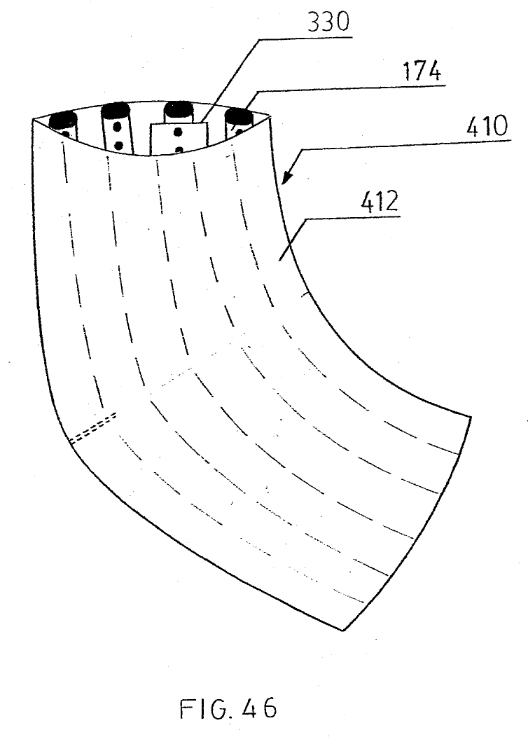

Shown in FIG. 46 is another embodiment wherein the venting device is shown with a substantial bend located centrally. The venting device can also be made straight for its entire length. The device 410 includes a stretchable piece of porous fabric material 412 having at least some elasticity and preferably a significant amount of elasticity in order to permit the device to be slid over and positioned on a person's or animal's limb that has been broken. Padding 330 may be added to the tubing 174 in any manner discussed above. Any suitable material may be used for the devices of FIGS. 44, 45 and 46 including Surgilast net material.

It will be understood that the venting device of the invention is adapted for placement around a part of a human body or animal body prior to application of a surgical cast over this part of the body. The surgical cast itself can be of standard construction and made of plaster of paris, for example.

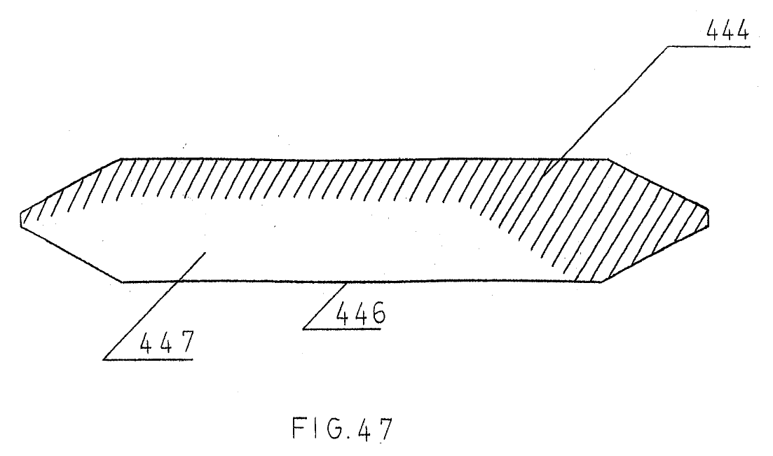

Furthermore, with respect to any of the above mentioned embodiments of cast venting devices, padding, preferably EVA or an EVA-PLASTAZOTE combination may be affixed over the entirety of the aerating devices (i.e. tubes, rings, squares, or any said aerating device described above) present on a venting strip, or it may be included on selected portion thereof. Furthermore, portions of the inner surface of such a strip may be selectively covered with the venting devices as desired, said devices themselves including or not including padding as desired. Selective placement of aerating devices and padding on a preferred version of a venting strip is shown in FIG. 47, which shows schematically aerating devices at 444, which could be any of the aerating devices disclosed herein, on a strip of fabric 446. A significant portion 447 of the strip does not include aerating devices thereupon. As desired, portions of the aerating devices may including padding affixed thereto. To enhance comfort of the user, aerating devices are preferably positioned adjacent and in close contact with each other, however they may be spaced apart from one another.

It will be appreciated by those skilled in the art that various modifications and changes can be made to the described and illustrated surgical cast venting devices without departing from the spirit and scope of the invention.

I claim:

1. A surgical cast venting device comprising:
a fabric sheet, having two opposite ends defining a length therebetween and having inner and outer surfaces, said sheet being applicable to a part of a human or animal body prior to application of a surgical cast over said body part, said inner surface facing said body part when said sheet is applied thereto;
a plurality of aerating devices affixed to said inner surface; and
padding material affixed to said aerating devices and positioned such that said padding material is disposed between said aerating devices and said body part when said sheet is applied to said body part.

2. A surgical cast venting device according to claim 1 wherein said aerating devices comprise a substantial number of tubular members each having two ends, and each being open ended at at least one end thereof.

3. A surgical cast venting device according to claim 2 wherein said tubular members are adhesively bonded to said fabric sheet, and extend in a longitudinal direction relative to said fabric sheet, said sheet comprising an elongate strip having a length extending from one end thereof to the opposite end.

4. A venting device according to claim 2 wherein each tubular member has at least several perforation holes formed through the side thereof and distributed along the length thereof.

5. A venting device according to claim 1 wherein said fabric sheet has substantial elasticity and a substantial tubular configuration.

6. A venting device according to claim 2 wherein said fabric sheet has substantial elasticity and a substantially tubular configuration and said tubular members are arranged in an elicoidal configuration on said inner surface.

7. A surgical cast venting device according to claim 1 wherein said padding material comprises ethyl vinyl acetate (EVA).

8. A surgical cast venting device according to claim 7 wherein said EVA has a hardness of Shore A equal to 18.

9. A surgical cast venting device according to claim 1 wherein said padding material comprises ethyl vinyl acetate (EVA) LUNAIMED.

10. A surgical cast venting device according to claim 4 wherein said padding material is a perforated ethyl vinyl acetate (EVA) material and at least some of the perforations in said EVA material align with said perforation holes in said tubular members.

11. A surgical cast venting device comprising:
a stretchable piece of porous fabric material having elasticity and substantially tubular configuration; said piece having an inner surface and being adapted to stretch and snugly fit over a body part prior to the application of a surgical cast, said piece having two opposite ends;
at least one layer of a plurality of flexible tubular members, distributed over, and affixed to said inner surface in an elicoidal pattern, said tubular members being perforated and open ended; and
padding material affixed to at least some of said tubular members such that said padding material is disposed between said tubular members and said body part.

12. A surgical cast venting device according to claim 11 wherein said padding material comprises ethyl vinyl acetate (EVA) and is elicoidally wrapped around said tubular members.

13. A surgical cast venting device according to claim 12 wherein said padding material has perforations and at least some of said perforations in said padding material align with some of the perforations in said tubular members.

14. A surgical cast venting device comprising:
a porous piece of fabric material having at least some elasticity and a substantially tubular configuration, said piece having two opposite ends, and an inner surface;
at least several, separate elongate plastic tubular members distributed over and affixed to a portion of said inner surface, said tubular members each being open ended at at least one end and having at least several ventilation holes formed in the tubular side thereof, said tubular members extending substantially from one of said opposite ends to the other of said opposite ends; and
padding material affixed to portions of at least some of said tubular members, said padding material including perforation holes therethrough, some of which being aligned with said ventilation holes;
wherein said venting device is adapted for placement around part of a human body or animal body prior to application of a surgical cast over said part of the body.

15. A surgical cast venting device according to claim 14 wherein said padding material comprises ethyl vinyl acetate (EVA).

16. A surgical cast venting device according to claim 15 wherein said padding material is wrapped around the length of said tubular members elicoidally.

17. A surgical cast venting device comprising:
an elongate flexible strip of porous fabric material for winding around part of a human or animal body prior to application of a surgical cast;
a number of elongate flexible tubing members distributed over and attached to said elongate strip on a portion of one side thereof; and
padding material attached to said tubing members and positioned such that said padding is disposed between tubing members and said body part when said elongate strip is wound around said body part.

18. A surgical cast venting device according to claim 17 wherein said elongate strip is tapered at opposite ends thereof and said tubes are attached at a small acute angle to a transverse line perpendicular to a longitudinal centreline of the elongate strip.

19. A surgical cast venting device according to claim 2 wherein said tubular members are positioned adjacent to each other.

20. A surgical cast venting device according to claim 2 wherein said padding material is in the form of a plurality of padding envelopes, each padding envelope defining a hollow central portion wherein groups of tubular members may be inserted.

21. A surgical cast venting device according to claim 1 including a thin layer of heat managing and perspiration absorbing material which is disposed between said padding material and said body part during use of the venting device.

22. A surgical cast venting device according to claim 21 wherein said thin layer of heat managing and perspiration absorbing material is Comfortem (TM) foam.
